(19) United States
(12) Patent Application Publication
(10) Pub. No.: US 2006/0128944 A1
(43) Pub. Date: Jun. 15, 2006

(54) Molecular linkers suitable for crystallization and structural analysis of molecules of interest, method of using same, and methods of purifying g protein-coupled receptors

(75) Inventors: Simone A. Botti, Rehovot (IL); Joel L. Sussman, Rehovot (IL); Israel Silman, Rehovot (IL); Terence Lewis, Berkshire (IL)

Correspondence Address: G E Ehrlich (1995);Antonhy Castorina, 2001 Jefferson Davis Highway, Suite 207, Arlington, VA, 22202, US

(73) Assignee: YEDA RESEARCH AND DEVELOPMENT CO. LTD. AT THE WEIZMANN INSTRITUTE OF SCIENCE

(21) Appl. No.: 10/485,828

(22) PCT Filed: Aug. 21, 2002
(86) PCT No.: PCT/IL02/00692

§ 371 Date: Aug. 9, 2004

(30) Foreign Application Priority Data
Aug. 21, 2001 (IL) 145035

(51) Int. Cl. C07K 14/705; G06F 19/00

(52) U.S. Cl. 530/350; 702/19

(57) ABSTRACT

A method of crystallizing a molecule-of-interest is disclosed. The method comprises (a) contacting molecules of the molecule-of-interest with at least one type of heterologous molecular linker being capable of interlinking at least two molecules of said molecule-of-interest to thereby form a crystallizable molecular complex of defined geometry; and (b) subjecting said crystallizable molecular complex to crystallization-inducing conditions, thereby generating the crystal containing said molecule-of-interest.

### FIELD AND BACKGROUND OF THE INVENTION

The present invention relates to molecular linkers suitable for crystallization and structural analysis of molecules of interest, to method of using same, and to methods of purifying G protein coupled receptors (GPCRs). More particularly, the present invention relates to methods of crystallizing membrane proteins and to methods of purifying GPCRs via affinity chromatography using arrestin derived polypeptides.

Importance of protein structure determination: The recently fully sequenced human genome, has been found to contain up to 38,000 genes (Venter J C. et al., 2001. Science 291:1304) encoding up to an order of magnitude more protein species. It is evident that the information contained therein holds tremendous potential for furthering the development of practical applications in all fields involving the life sciences. However, most proteins remain to be characterized with respect to their structure and function and, although the transcription profiles of the genes encoding these proteins are currently being determined, such data can yield only limited information. In order to fully harness the potential of the information contained in the complete human genome sequence, it will be necessary to systematically determine the three-dimensional (3D) structure of the proteins encoded therein.

The capacity to solve the 3D atomic structure of proteins is proving to be crucial for understanding and regulating their biological functions and, as such, is playing an increasingly vital role in the advancement of biomedical science and biotechnology, in particular in the realm of drug design.

The pathogenesis of a very large number of human diseases involves membrane proteins such as GPCRs, as startlingly demonstrated by the fact that a 60% majority of approved drugs elicit their therapeutic effects by selectively targeting members of the GPCR family (GlaxoWellcome, 1996. Nature Suppl. 384:1-5). However, pharmacological treatment of diseases involving GPCRs remains far from optimal and there is thus a critical need for novel and improved GPCR-targeting drugs. As highlighted, for example, by the 3D atomic structure-based development of protease inhibitors employed in the first effective treatment of human immunodeficiency virus (HIV) induced acquired immuno-deficiency syndrome (AIDS) (Wlodawer A. and Vondrasek J., 1998. Annu Rev Biophys Biomol Struct. 27:249), the development of novel and improved membrane protein-targeting drugs, such as GPCR-targeting drugs, can dramatically benefit from the availability of the 3D atomic structure of such drug targets.

Other increasingly important applications of protein crystals include their use as catalysts on a commercial scale, in bioremediation and green chemistry applications, and in purification-related applications, such as enantioselective chromatography of pharmaceuticals and high-grade chemicals. In the near future, their utility will further expand to include the purification of protein drugs and the development of adjuvant-less vaccines (Margolin A L. and Navia M A., 2001. Angewandte Chemie International Edition 40:2204).

General obstacles to protein crystallization: The bottleneck in determination of novel protein structures has shifted from the collection and interpretation of crystallographic data to the production of large amounts of highly pure protein and the generation of diffraction-grade crystals. Techniques for growing such crystals currently rely substantially on empirical processes for which only general rules of thumb are available and which frequently require adaptations tailored to accommodate the peculiarities of individual proteins.

Several factors contribute to the difficulty in obtaining highly ordered protein crystals. Although contacts between crystallized protein molecules are of comparable energy to those between small molecules, the significantly fewer number of intermolecular contacts per molecular weight of crystallized protein molecules renders these contacts very fragile (Carugo O. and Argos P., 1997. Protein Science 6:2261). Furthermore, due to their inherent complexity, protein molecules can assume numerous conformations, a phenomenon which tends to prevent formation of highly ordered crystals. Moreover, aggregated proteins are able to form many different types of intermolecular contacts of which only a restricted number will generate highly ordered crystals. Hence, crystallization conditions must be carefully fine-tuned so as to induce the proper molecular conformation and packing orientation of each molecule accreted during the process of crystallization. Such conditions are difficult to obtain since small variations in physico-chemical parameters, such as pH, ionic strength, temperature or contaminants, will strongly influence the process of crystallization in a way that is unique for each protein due to the diversity of the chemical groups and possible configurations thereof involved in the formation of intermolecular contacts (Giege R. et al., Acta Crystallographica Section D-Biological Crystallography 1994. 50:339; Durbin S D. and Feher G., 1996. Annu Rev Phys Chem. 47:171; Weber P C., Overview of protein crystallization methods, in Macromolecular Crystallography, Pt a. 1997. p. 13-22; Chernov A A., Physics Reports-Review Section of Physics Letters 1997. 288:61; Rosenberger F., Theoretical and Technological Aspects of Crystal Growth 1998. p. 241; Wiencek J M., 1999. Annu Rev Biomed Eng. 1:505).

Obstacles to Membrane Protein Crystallization

Three dimensional protein structure determination at high resolution represents a particularly difficult challenge for membrane proteins and the number of such proteins that have been crystallized is still small and far behind that of soluble proteins, even though membrane proteins represent up to 40% of the proteins encoded by the human genome (Wallin E. and von Heijne G., 1998. Protein Sci. 7:1029).

The crystallization of membrane proteins is particularly difficult due to the fact that, unlike soluble proteins which tend to have hydrophilic surfaces and polar cores, membrane proteins have significant hydrophobic surfaces through which they interact with membrane lipids. Such proteins exist in a quasi-solid state in the membrane and are not readily soluble in either aqueous or apolar environments.

The most widely employed approach for solubilization of membrane proteins is the use of detergents interacting with the hydrophobic surfaces of the protein to generate mixed detergent/protein micelles. Solubilized membrane proteins can then be crystallized in an ordered two-dimensional (2D) lattice by reconstitution in an artificial lipid bilayer, allowing 2D structural determination via electron microscopy. While such 2D crystals are relatively easy to obtain, the use of electron microscopy for determining molecular structure suffers from the significant drawback of generating structural information with poor resolution in directions orthogonal to the 2D lattice, thus preventing structural determination at sufficiently high resolutions (Stowell M H. et al., 1998. Curr Opin Struct Biol. 8:595). An additional factor contributing to the difficulty of determining the structure of membrane proteins at high resolution is due to the fact that crystal contacts made between detergent micelles tend to be disordered, resulting in poorly diffracting crystals. Although the use of helical crystals and advanced image processing can obviate some of these drawbacks, it is only with X-ray crystallography of 3D crystals that high resolution determination of 3D protein structure can be achieved. This is essential, for example, to generate detailed pictures of molecular target sites when designing drugs specifically interacting with such sites. In the case of membrane proteins, this is highly desirable since such information can significantly contribute to the design and development of novel drugs for the very large number of diseases whose pathogenesis involves membrane proteins, such as receptors. Such diseases include, for example, cancer, viral diseases such as AIDS, neurological disorders, metabolic illnesses such as diabetes, etc.

Prior Art Optimization of Crystallization Conditions

High Throughput Techniques

High throughput techniques are currently being employed to determine the conditions required for growth of protein crystals. One such approach employs automation to perform large numbers of crystallization trials (Morris, D W. et al., 1989. Biotechniques 7:522; Zuk W M. and Ward K B., 1991. Journal of Crystal Growth 110:148; Heinemann U. et al., 2000. Progress in Biophysics & Molecular Biology 73:347).

Such high throughput approaches employ the sparse-matrix protein crystallization method, in which a series of crystallization conditions are tested in parallel, the most promising ones being iteratively refined until crystallization is achieved (Jancarik J. and Kim S H., 1991. Journal of Applied Crystallography 24:409; Cudney B., et al., 1994. Acta Crystallographica Section D-Biological Crystallography 50:414; Hennessy D. et al., 2000. Acta Crystallographica Section D—Biological Crystallography 56:817).

However, successful crystallization of membrane proteins via such techniques is highly inefficient due to the high tendency of membrane proteins to denature and/or aggregate during crystallization. Furthermore, such methods, being substantially empirical, present the disadvantages of being both time-consuming and of requiring large amounts of pure protein, a requirement which is generally difficult or expensive to fulfill.

One strategy which has been suggested in order to circumvent the disadvantages inherent to such high throughput techniques is to assist the crystallization of molecules which are otherwise difficult or impossible to crystallize by either modifying such molecules so as to facilitate their crystallization, or by crystallizing such molecules in complex with other molecules susceptible to provide an ordered matrix facilitating formation of the basic unit of a crystal lattice.

Protein-modification techniques: One approach attempting to improve membrane protein crystal growth and ordering has employed complexation of a protein of interest with antibody fragments prior to crystallization (Hunte C., 2001. FEBS Lett. 504:126-32; Lange C. & Hunte C., 2002. Proc Natl Acad Sci USA. 99:2800-5; Ostermeier C. and Michel H., 1997. Curr Opin Struct Biol. 7:697; Ostermeier C. et al., 1997. Proc Natl Acad Sci USA. 94:10547-53).

Another modification based approach has used fusion of proteins to be crystallized to large hydrophobic domains derived from heterologous proteins in an attempt to minimize the overall hydrophobicity of proteins of interest (Prive G. et al., 1994. Biol Crystallogr. D50:375).

Yet another approach involves alteration and engineering of crystal unit cell contacts, an example being the crystallization of apoferritin by site-directed mutagenesis of residues involved in the binding of a Co²⁺ atom introduced during the crystallization process (Takeda S. et al., 1995. Proteins, 23:548).

These approaches, however, have the significant drawback that identifying and creating suitable fusion proteins or engineering residues involved in crystal contacts are ad hoc and very labor intensive procedures requiring much fine tuning for applicability to any given protein.

Functionalized lipids: Still another approach has employed binding of functionalized lipids to proteins of interest in an attempt to generate crystalline arrays of such proteins. For example, divalent metal ion-chelated lipids or electrostatically charged lipids have been employed to bind proteins via specific surface histidine residues or via complementarily charged residues, respectively. The use of planar layers of such lipids has been employed to generate 2D crystals (Frey W. et al., Proc Nat Acad Sci. USA 1996 93:4937) which can be studied by electron microscopy, but not by X-ray diffraction, thereby yielding limited structural information in terms of dimensionality and in terms of resolution.

A more advanced variant of this approach has utilized lipid nanotubes to generate helical crystals (Wilson-Kubalek, E. et al., Proc. Natl. Acad. Sci. U.S.A. 1998, 95:8040). These crystals, however, can only be used to determine 3D protein structure at low resolution using electron microscopy and thus cannot be employed to solve molecular structure at atomic resolution, as is the case with X-ray crystallography.

Thus, all prior art approaches have failed to provide an adequate solution for efficiently generating X-ray diffraction grade crystals of molecules such as membrane proteins.

There is thus a widely recognized need for and it would be highly advantageous to have, a method of crystallizing molecules, such as membrane proteins, devoid of the above limitations.

### SUMMARY OF THE INVENTION

According to one aspect of the present invention there is provided a method of generating a crystal containing a molecule-of-interest, the method comprising: (a) contacting molecules of the molecule-of-interest with at least one type of heterologous molecular linker being capable of interlinking at least two molecules of the molecule-of-interest to thereby form a crystallizable molecular complex of defined geometry; and (b) subjecting the crystallizable molecular complex to crystallization-inducing conditions, thereby generating the crystal containing the molecule-of-interest.

According to further features in preferred embodiments of the invention described below, the at least one type of heterologous molecular linker is selected such that the crystallizable molecular complex formed is capable of generating a crystal selected from the group consisting of a 2D crystal, a helical crystal and a 3D crystal.

According to still further features in preferred embodiments, the molecule-of-interest is a polypeptide.

According to still further features in preferred embodiments, the polypeptide is a membrane protein.

According to still further features in preferred embodiments, the membrane protein is a G protein coupled receptor.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the at least one type of heterologous molecular linker includes a region for specifically binding the molecule-of-interest.

According to still further features in preferred embodiments, the molecule-of-interest is a G protein coupled receptor and the region for specifically binding the molecule-of-interest comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the molecule-of-interest includes a histidine tag and the region for specifically binding the molecule-of-interest comprises a nickel ion or an antibody specific for the histidine tag.

According to still further features in preferred embodiments, the molecule-of-interest includes core streptavidin and the region for specifically binding the molecule-of-interest comprises a biotin moiety or a Strep-tag.

According to still further features in preferred embodiments, the molecule-of-interest includes a biotin moiety or a Strep-tag and the region for specifically binding the molecule-of-interest comprises core streptavidin.

According to still further features in preferred embodiments, the molecule-of-interest is a G protein coupled receptor and the at least one type of molecular linker comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the at least one type of heterologous molecular linker includes at least two non-covalently bound subunits.

According to still further features in preferred embodiments, the at least two non-covalently bound subunits comprise a first subunit comprising a homomultimerizing portion and a metal-binding portion, and a second subunit comprising a portion specifically binding the molecule-of-interest, According to still further features in preferred embodiments, the at least two non-covalently bound subunits comprise a first subunit comprising a homomultimerizing portion and a portion specifically binding the molecule-of-interest, and a second subunit comprising a metal-binding portion, and a portion specifically binding the first subunit.

According to still further features in preferred embodiments, the at least one type of heterologous molecular linker includes a molecule selected from the group consisting of a polycyclic molecule, a polydentate ligand, a macrobicyclic cryptand, a polypeptide and a metal.

According to still further features in preferred embodiments, the at least one type of heterologous molecular linker comprises core streptavidin.

According to still further features in preferred embodiments, the at least one type of heterologous molecular linker is selected so as to define the spatial positioning and orientation of the at least two molecules within the crystallizable molecular complex, thereby facilitating crystallization of the molecule-of-interest.

According to still further features in preferred embodiments, the at least one type of heterologous molecular linker includes a hydrophilic region, the hydrophilic region being for facilitating crystallization of the molecule-of-interest.

According to still further features in preferred embodiments, the at least one type of heterologous molecular linker includes a conformationally rigid region, the conformationally rigid region being for facilitating crystallization of the molecule-of-interest.

According to still further features in preferred embodiments, the at least one type of heterologous molecular linker includes a metal-binding moiety capable of specifically binding a metal atom, the metal atom being capable of facilitating crystallographic analysis of the crystal.

According to still further features in preferred embodiments, the metal-binding moiety is a metal binding protein.

According to still further features in preferred embodiments, the metal binding protein is metallothionein.

According to still further features in preferred embodiments, the at least one type of heterologous molecular linker includes a region being capable of functioning as a purification tag, the purification tag being capable of facilitating purification of the crystallizable molecular complex and/or of facilitating the interlinking at least two molecules of the molecule-of-interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the molecule-of-interest includes a region being capable of functioning as a purification tag, the purification tag being capable of facilitating purification of the crystallizable molecular complex, and/or of facilitating the interlinking at least two molecules of the molecule-of-interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the molecule-of-interest includes a metal-binding moiety capable of specifically binding a metal atom, the metal atom being capable of facilitating crystallographic analysis of the crystal.

According to still further features in preferred embodiments, the metal-binding moiety is a metal binding protein.

According to still further features in preferred embodiments, the metal binding protein is metallothionein.

According to another aspect of the present invention there is provided a method of generating a crystal containing a polypeptide of interest, the method comprising: (a) providing a molecule including the polypeptide of interest and a heterologous multimerization domain being capable of directing the homomultimerization of the polypeptide of interest; (b) subjecting the molecule to homomultimerization-inducing conditions, thereby forming a crystallizable molecular complex; and (c) subjecting the crystallizable molecular complex to crystallization-inducing conditions, thereby generating the crystal containing the polypeptide of interest.

According to further features in preferred embodiments of the invention described below, steps (a) and (b) are effected concomitantly.

According to still further features in preferred embodiments, the heterologous multimerization domain is selected such that the crystallizable molecular complex formed is capable of generating a crystal selected from the group consisting of a 2D crystal, a helical crystal and a 3D crystal.

According to still further features in preferred embodiments, the heterologous multimerization domain includes a hydrophilic region, the hydrophilic region being for facilitating crystallization of the polypeptide of interest.

According to still further features in preferred embodiments, the heterologous multimerization domain includes a conformationally rigid region, the conformationally rigid region being for facilitating crystallization of the polypeptide of interest.

According to still further features in preferred embodiments, the heterologous multimerization domain is selected so as to define the spatial positioning and orientation of polypeptides of the polypeptide of interest within the crystallizable molecular complex, thereby facilitating crystallization of the polypeptide of interest.

According to still further features in preferred embodiments, the heterologous multimerization domain comprises core streptavidin.

According to still further features in preferred embodiments, the polypeptide of interest is a G protein coupled receptor and the heterologous multimerization domain comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the polypeptide of interest includes a histidine tag and the heterologous multimerization domain comprises a nickel ion or an antibody specific for the histidine tag.

According to still further features in preferred embodiments, the polypeptide of interest includes core streptavidin and the heterologous multimerization domain comprises a biotin moiety or a Strep-tag.

According to still further features in preferred embodiments, the polypeptide of interest includes a biotin moiety or a Strep-tag and the heterologous multimerization domain comprises core streptavidin.

According to still further features in preferred embodiments, the polypeptide of interest and the heterologous multimerization domain are interlinked via a molecular linker.

According to still further features in preferred embodiments, at least one of the heterologous multimerization domain and the molecular linker include a hydrophilic region, the hydrophilic region being for facilitating crystallization of the polypeptide of interest.

According to still further features in preferred embodiments, at least one of the heterologous multimerization domain and the molecular linker include a conformationally rigid region, the conformationally rigid region being for facilitating crystallization of the polypeptide of interest.

According to still further features in preferred embodiments, at least one of the heterologous multimerization domain and the molecular linker is selected so as to define the spatial positioning and orientation of polypeptides of the polypeptide of interest within the crystallizable molecular complex, thereby facilitating crystallization of the polypeptide of interest.

According to still further features in preferred embodiments, the at least one molecular linker includes a region being capable of functioning as a purification tag, the purification tag being capable of facilitating purification of the crystallizable molecular complex, and/or of facilitating the homomultimerization of the polypeptide of interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the polypeptide of interest includes a region being capable of functioning as a purification tag, the purification tag being capable of facilitating purification of the crystallizable molecular complex, and/or of facilitating the homomultimerization of the polypeptide of interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the molecule includes a metal-binding moiety capable of specifically binding a metal atom, the metal atom being capable of facilitating crystallographic analysis of the crystal.

According to still further features in preferred embodiments, the metal-binding moiety is a metal binding protein.

According to still further features in preferred embodiments, the metal binding protein is metallothionein.

According to still further features in preferred embodiments, the polypeptide of interest is a membrane protein.

According to still further features in preferred embodiments, the membrane protein is a G protein coupled receptor.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the polypeptide of interest includes a metal-binding moiety capable of specifically binding a metal atom, the metal atom being capable of facilitating crystallographic analysis of the crystal.

According to still further features in preferred embodiments, the metal binding moiety is metallothionein.

According to yet another aspect of the present invention there is provided a composition-of-matter comprising at least two molecules of a molecule-of-interest interlinked via a heterologous molecular linker, wherein the heterologous molecular linker is selected so as to define the relative spatial positioning and orientation of the at least two molecules within the composition-of-matter, thereby facilitating formation of a crystal therefrom under crystallization-inducing conditions.

According to further features in preferred embodiments of the invention described below, the molecule-of-interest is a polypeptide.

According to still further features in preferred embodiments, the polypeptide is a membrane protein.

According to still further features in preferred embodiments, the membrane protein is a G protein coupled receptor.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the heterologous molecular linker includes at least one region capable of specifically binding the molecule-of-interest.

According to still further features in preferred embodiments, the molecule-of-interest is a G protein coupled receptor and the at least one region capable of specifically binding the molecule-of-interest is a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the heterologous molecular linker includes a molecule selected from the group consisting of a polycyclic molecule, a polydentate ligand, a macrobicyclic cryptand, a polypeptide and a metal.

According to still further features in preferred embodiments, the molecule-of-interest is a G protein coupled receptor and the heterologous molecular linker comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the heterologous molecular linker comprises core streptavidin.

According to still further features in preferred embodiments, the heterologous molecular linker includes at least two non-covalently bound subunits.

According to still further features in preferred embodiments, the heterologous molecular linker includes a hydrophilic region, the hydrophilic region being for facilitating crystallization of the molecule-of-interest.

According to still further features in preferred embodiments, the heterologous molecular linker includes a conformationally rigid region, the conformationally rigid region being for facilitating crystallization of the molecule-of-interest.

According to still further features in preferred embodiments, the heterologous molecular linker is selected such that the composition-of-matter is capable of generating a crystal selected from the group consisting of a 2D crystal, a helical crystal and a 3D crystal.

According to still further features in preferred embodiments, the heterologous molecular linker includes a metal-binding moiety capable of specifically binding a metal atom, the metal atom being capable of facilitating crystallographic analysis of the crystal.

According to still further features in preferred embodiments, the metal-binding moiety is a metal-binding protein.

According to still further features in preferred embodiments, the metal binding protein is metallothionein.

According to still further features in preferred embodiments, the heterologous molecular linker includes a region being capable of functioning as a purification tag, the purification tag being capable of facilitating purification of the crystallizable composition-of-matter, and/or of facilitating the interlinking of the at least two molecules of a molecule-of-interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the molecule-of-interest includes a region being capable of functioning as a purification tag, the purification tag being capable of facilitating purification of the composition-of-matter, and/or of facilitating the interlinking of the at least two molecules of a molecule-of-interest.

According to still further features in preferred embodiments, the region being capable of functioning as a purification tag is selected from the group consisting of a T7 tag, a histidine tag, a Strep-tag, core streptavidin, and biotin.

According to still further features in preferred embodiments, the molecule-of-interest includes a metal-binding moiety capable of specifically binding a metal atom, the metal atom being capable of facilitating crystallographic analysis of the crystal.

According to still further features in preferred embodiments, the metal-binding moiety is a metal binding protein.

According to still further features in preferred embodiments, the metal-binding protein is metallothionein.

According to still another aspect of the present invention there is provided a nucleic acid construct comprising a polynucleotide segment encoding a chimeric polypeptide including: (a) a first polypeptide region being capable of specifically binding a molecule-of-interest; and (b) a second polypeptide region being capable of specifically binding a metal atom.

According to further features in preferred embodiments of the invention described below, the molecule-of-interest is a G protein coupled receptor and the chimeric polypeptide comprises SEQ ID NO: 5 or SEQ ID NO: 6.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the molecule-of-interest is a G protein coupled receptor and the first polypeptide region comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the molecule-of-interest is a polypeptide.

According to still further features in preferred embodiments, the polypeptide is a membrane protein.

According to still further features in preferred embodiments, the membrane protein is a G protein coupled receptor.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the second polypeptide region is metallothionein.

According to still further features in preferred embodiments, the chimeric polypeptide is selected such that when combined with molecules of the molecule-of-interest under suitable conditions, the chimeric polypeptide and the molecules form a crystallizable molecular complex which is capable of forming a crystal containing the molecule-of-interest when subjected to crystallization-inducing conditions.

According to still further features in preferred embodiments, the chimeric polypeptide is selected such that when combined with molecules of the molecule-of-interest and the metal atom under suitable conditions, the chimeric polypeptide and the molecules form a crystallizable molecular complex which is capable of forming a crystal containing the molecule-of-interest when subjected to crystallization-inducing conditions.

According to still further features in preferred embodiments, the metal atom facilitates crystallographic analysis of the crystal.

According to still further features in preferred embodiments, the chimeric polypeptide includes a hydrophilic region, the hydrophilic region being for facilitating crystallization of the molecule-of-interest.

According to still further features in preferred embodiments, the chimeric polypeptide includes a conformationally rigid region, the conformationally rigid region being for facilitating crystallization of the molecule-of-interest.

According to still further features in preferred embodiments, the chimeric polypeptide is selected so as to define the spatial positioning and orientation of the molecule-of-interest within the crystallizable molecular complex, thereby facilitating crystallization of the molecule-of-interest.

According to still further features in preferred embodiments, the chimeric polypeptide is selected such that the crystallizable molecular complex formed is capable of generating a crystal selected from the group consisting of a 2D crystal, a helical crystal and a 3D crystal.

According to still further features in preferred embodiments, the chimeric polypeptide further includes a polypeptide region being capable of functioning as a purification tag, the purification tag being capable of facilitating purification of the crystallizable molecular complex, and/or of facilitating the binding of a molecule-of-interest.

According to still further features in preferred embodiments, the region being capable of functioning as a purification tag is selected from the group consisting of a T7 tag, a histidine tag, a Strep-tag, core streptavidin, and biotin.

According to a further aspect of the present invention there is provided a nucleic acid construct comprising a polynucleotide segment encoding a chimeric polypeptide including: (a) a first polypeptide region being capable of specifically binding a molecule-of-interest; (b) a second polypeptide region being capable of homomultimerization into a complex of defined geometry; and (c) a third polypeptide region being capable of specifically binding a metal atom.

According to further features in preferred embodiments of the invention described below, the molecule-of-interest is a G protein coupled receptor and the first polypeptide region is selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

According to still further features in preferred embodiments, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the second polypeptide region comprises core streptavidin.

According to still further features in preferred embodiments, the molecule-of-interest is a G protein coupled receptor and the chimeric polypeptide comprises SEQ ID NO: 5 or SEQ ID NO: 6.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the third polypeptide region comprises metallothionein.

According to still further features in preferred embodiments, the molecule-of-interest is a polypeptide.

According to still further features in preferred embodiments, the polypeptide is a membrane protein.

According to still further features in preferred embodiments, the membrane protein is a G protein coupled receptor.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the chimeric polypeptide is selected such that when combined with molecules of the molecule-of-interest, the chimeric polypeptide and the molecules form a crystallizable molecular complex of defined geometry which is capable of forming a crystal containing the molecule-of-interest when subjected to crystallization-inducing conditions.

According to still further features in preferred embodiments, the chimeric polypeptide includes a hydrophilic region, the hydrophilic region being for facilitating crystallization of the molecule-of-interest.

According to still further features in preferred embodiments, the chimeric polypeptide includes a conformationally rigid region, the conformationally rigid region being for facilitating crystallization of the molecule-of-interest.

According to still further features in preferred embodiments, the chimeric polypeptide is selected so as to define the spatial positioning and orientation of molecules of the molecule-of-interest within the crystallizable molecular complex, thereby facilitating crystallization of the molecule-of-interest.

According to still further features in preferred embodiments, the chimeric polypeptide is selected such that the crystallizable molecular complex of defined geometry formed is capable of generating a crystal selected from the group consisting of a 2D crystal, a helical crystal and a 3D crystal.

According to still further features in preferred embodiments, the metal atom facilitates crystallographic analysis of the molecule-of-interest contained in the crystal.

According to still further features in preferred embodiments, the chimeric polypeptide further includes a polypeptide region being capable of functioning as a purification tag, the purification tag being capable of facilitating purification of the crystallizable molecular complex, and/or of facilitating the binding of a molecule-of-interest.

According to still further features in preferred embodiments, the region being capable of functioning as a purification tag is selected from the group consisting of a T7 tag, a histidine tag, a Strep-tag, and core streptavidin.

According to a yet a further aspect of the present invention there is provided a method of purifying a G protein coupled receptor from a sample containing the G protein coupled receptor, the method comprising subjecting the sample to affinity chromatography using an affinity ligand selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, a molecule defined by SEQ ID NO: 3, and a molecule defined by SEQ ID NO: 4, thereby purifying the G protein coupled receptor.

According to further features in preferred embodiments of the invention described below, the at least a portion of an arrestin molecule is homologous to amino acid residues 11 to 190, or 11 to 370 of human beta-arrestin-1a.

According to still further features in preferred embodiments, the at least a portion of an arrestin molecule comprises a G protein coupled receptor-binding domain of the arrestin molecule.

According to still further features in preferred embodiments, the mutation at an amino acid residue position corresponding to position 90 in bovine visual arrestin is a mutation to a serine or threonine residue.

According to still further features in preferred embodiments, the mutation at an amino acid residue position corresponding to position 175 in bovine visual arrestin is a mutation to a glutamic acid or an asparagine residue.

According to still further features in preferred embodiments, the G protein coupled receptor is rhodopsin or is a class A G protein coupled receptor.

According to still further features in preferred embodiments, the class A G protein coupled receptor is m2 muscarinic cholinergic receptor.

According to still further features in preferred embodiments, the affinity ligand includes a region being capable of functioning as a purification tag, the purification tag being capable of facilitating attachment of the affinity ligand to an affinity chromatography matrix.

According to still further features in preferred embodiments, the region being capable of functioning as a purification tag is selected from the group consisting of a T7 tag, a histidine tag, a Strep-tag, core streptavidin, and biotin.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a diagram depicting the general configuration of a non-polypeptidic molecular linker which can be used for multimerization of a molecule-of-interest according to the teachings of the present invention. MS: molecular scaffold, M: metal atom; L: linking chain containing 1-3 carbon or oxygen atoms (shown in FIG. 1b); G=[—CO₂], [—CONH], [—O], [—OCO] or [—NHCO]; L′=linking chain of 1-10 atoms containing carbon or oxygen atoms, such as [(CH₂CH₂O)₂—O—CH₂CH₂—] or [—(CH₂)₄—]; SBD=specific binding domain, such as [—N⁺(CH₃)₃] or [—CO(CF₃)], or a polypeptide such as biotin.

FIG. 1b is a diagram depicting a linking chain containing 1-3 carbon or oxygen atoms comprised in the non-polypeptidic molecular linker described in FIG. 1a. G′=[CO₂H], [OH] or [NH₂].

FIGS. 2a-b are diagrams depicting porphyrin-based molecular linkers which can be used according to the teachings of the present invention for multimerization of two (FIG. 2a) or four (FIG. 2b) molecules of interest. X=[L-G-L′-SBD], as defined in FIG. 1a; R=H, (sub)-phenyl or [L-G-L′-SBD], as defined in FIG. 1a, M=metal atom.

FIG. 3 is a diagram depicting a hydroxime-based molecular linker which can be used according to the teachings of the present invention for multimerization of two molecules of interest. X=[L-G-L′-SBD], R′=H, (sub)-phenyl or [L-G-L′-SBD], as defined in FIG. 1a; R′=H or methyl group; M=metal atom.

FIGS. 4a-b are schematic diagrams depicting synthesis of the porphyrin molecular linkers of FIGS. 2a-b which can be used for multimerization of four (FIG. 4a) or two (FIG. 4b) molecules of interest. HY=a strong acid; MZ₂=a transition or heavy metal salt; Oxid=an oxidant, such as DDQ or O₂.

FIG. 5 is a schematic diagram depicting synthesis of the hydroxime-based molecular linker of FIG. 3. MZ₂=a transition or heavy metal salt.

FIG. 6a is a schematic diagram depicting linkage of a biotinylated moiety to porphyrin-based molecular linkers such as those depicted in FIGS. 2a-b.

FIG. 6b is a schematic diagram depicting linkage of a trimethylammonium moiety to hydroxime-based molecular linkers such as the one depicted in FIG. 3. MZ₂=a transition or heavy metal salt.

FIGS. 7a-b are schematic diagrams depicting polynucleotide constructs for purification of molecules of interest. FIG. 7a is a diagram depicting a construct encoding a chimeric polypeptide containing a single-chain Fv (scFv) segment fused to a core streptavidin and purification tag segments. FIG. 7b is a diagram depicting a construct encoding a chimeric polypeptide containing a Strep-tag (Stag) segment fused to a metal atom binding polypeptide (MBP) segment fused in turn to a purification tag segment. The relative positions of the Strep-tag and metal atom binding polypeptide can also be inverted. NH₂-amino-terminus; leader-leader sequence or signal peptide for expression in eukaryotic or prokaryotic cells; V_{H} and V_{L}-antibody variable heavy and light chains, respectively.

FIG. 8 is a diagram depicting the conformation of a core-streptavidin tetramer used in the molecular linkers of the present invention indicating the N-terminal fusion sites thereof for attachment of moieties capable of specifically binding a molecule-of-interest, such as a single-chain Fv, and the binding site for attachment of a Strep-tag or a biotin moiety.

FIGS. 9a-b are sequence diagrams depicting the amino acid residue sequence of portions of human beta-arrestin-1a suitable for binding different classes of GPCRs with high affinity and specificity independently of the phosphorylation-activation state thereof. FIG. 9a depicts a polypeptide composed of amino acid residues 11-190 of human beta-arrestin-1a with mutation R169E. FIG. 9b depicts a polypeptide composed of amino acid residues 11-370 of human beta-arrestin-1a with mutation R169E. In both polypeptides, mutation R169E conferring the capacity to bind GPCRs independently of the phosphorylation-activation state thereof, and the wild type serine residue at position 86 conferring the capacity to bind multiple types of GPCRs are indicated (bold underlined).

FIGS. 10a-b are sequence diagrams depicting the amino acid residue sequence of molecular linkers for crystallization of different classes of GPCRs independently of the phosphorylation-activation state thereof. FIG. 10a depicts a linker composed of a chimeric protein consisting of the N- to C-terminal segments; T7 tag (N-terminal italics), core streptavidin (uppercase), the peptide linker GSAA (SEQ ID NO: 1; internal italics), and amino acid residues 11-190 of human beta-arrestin-1a (lowercase) with mutation R169E. FIG. 10b depicts a linker composed of a chimeric protein consisting of the N- to C-terminal segments; T7 tag (N-terminal italics), core streptavidin (uppercase), the peptide linker GSAA (SEQ ID NO: 1; internal italics), and amino acid residues 11-370 of human beta-arrestin-1a (lowercase) with mutation R169E. In the arrestin derived segment of both molecular linkers, mutation R169E conferring the capacity to bind GPCRs independently of the phosphorylation-activation state thereof, and the wild type serine residue at position 86 conferring the capacity to bind multiple types of GPCRs are indicated (bold underlined).

FIG. 11 is a chemical structure diagram depicting a porphyrin-NTA-Ni²⁺ molecular linker used for crystallization of histidine-tagged proteins.

### DESCRIPTION OF THE PREFERRED EMBODIMENTS

The present invention is of methods and compositions which can be used for generating crystals containing a molecule-of-interest, and of methods of purifying G protein coupled receptors (GPCRs). Specifically, the present invention can be used to generate crystals of membrane proteins which can be used to determine the three-dimensional (3D) atomic structure thereof, and to purify GPCRs using arrestin derived polypeptides as affinity ligands of GPCRs.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Various methods of assisting the crystallization of molecules such as polypeptides and of facilitating their crystallographic analysis have been described in the prior art.

Techniques involving protein modifications, such as those based on fusion of the polypeptide of interest to a large heterologous hydrophobic polypeptide domain, alteration and engineering of crystal unit cell contacts or complexation of a protein of interest with antibody fragments are typically dedicated, labor intensive and require much fine tuning. In addition, methods relying on artificial functionalized lipid scaffolds are only useful for the creation of planar 2D crystals which can be studied by electron microscopy, but not by X-ray diffraction, or are useful for generation of helical crystals which do not permit high resolution 3D structural analysis.

Thus, prior art approaches for assisting or facilitating crystallization of molecules-of-interest have failed to provide adequate solutions for the controlled 3D crystallization of molecules such as polypeptides, while allowing subsequent determination of their 3D atomic structure.

In sharp contrast to prior art techniques, the methods of the present invention enable the generation of readily crystallizable molecular complexes incorporating molecules of a molecule-of-interest, such as a membrane protein. In addition, the present invention also enables purification of the molecule-of-interest, thereby greatly facilitating crystallographic analysis thereof.

Thus, according to the present invention, there is provided a method of generating a 2D, or preferably a 3D, crystal containing a molecule-of-interest.

According to one embodiment of the method of the present invention, crystallization of a molecule-of-interest is effected by contacting molecules of the molecule-of-interest with at least one type of linker. The linker is selected so as to be capable of interlinking at least two molecules of the molecule-of-interest to thereby form a crystallizable molecular complex of defined geometry (defined spatial orientation). As is further described hereinunder, the linker can be composed of a single molecule or a complex including a plurality of molecules, depending on the application and purpose.

Following linker-molecule-of-interest binding, the molecular complex formed is subjected to crystallization-inducing conditions, such as those described in Example 6 of the Examples section, thereby generating the crystal containing the molecule-of-interest.

As mentioned hereinabove, both single molecule and multi-molecule linker configurations can be used by the present invention.

A single-molecule linker can include binding regions covalently attached to a core, while a multi-molecule linker (linker complex) can include binding regions non-covalently associated with a core unit, and/or may include a core unit composed of non-covalently associated subunits. In any case, the linker is designed and configured such that when complexed with molecules of a molecule-of-interest, the linker directs the spatial orientation of the molecules of the molecule-of-interest so as to form a molecular complex of pre-defined geometry, thereby facilitating crystallization of the molecule-of-interest when the molecular complex is subjected to crystallization inducing conditions. The following Examples section describes specific examples of single-molecule and multi-molecule type linkers, as further detailed hereinbelow.

As used herein, a “core” of a linker refers to a portion of the linker functioning as the basic molecule-of-interest multimerization scaffold of the linker.

Regardless of core configuration, minimizing core size may be advantageous depending on the application and purpose. Cores of minimal size may be generally advantageous since this may minimize the size of the linker, which in turn serves to maximize tightness of packing of the molecular complex. This minimizes conformational disorder in the molecular complex, thus ensuring optimal ordering of crystals. As a further advantage, minimizing core size may make the linker easier and/or cheaper to produce and purify.

Single molecule linkers, being composed of covalently connected atoms, are highly stable and rigid and can be advantageously used to generate molecular complexes having minimized conformational disorder, for example, relative to linker complexes. Thus, single molecule linkers can be used to generate optimally ordered crystals, and may be more conveniently, cheaply, and/or easily produced relative to linker complexes.

Linker complexes may advantageously comprise homomultimerized proteins, such as, for example, fusion proteins comprising a homomultimerizing domain and a polypeptide or polypeptides, such as a binding domain and/or a purification tag, being capable of facilitating crystallization and/or 3D structure determination of a molecular complex, as further described hereinbelow. The use of linker complexes comprising such homomultimerized fusion proteins may be advantageously employed to obviate the need to separately express the polypeptide components of such fusion proteins, as well as the need to subject such components to conditions facilitating their association, thereby greatly facilitating generation of the linker complex, generation of the molecular complex, and/or crystallization of a molecule-of-interest.

The linkers of the present invention include one or preferably several binding domains for specifically binding the molecule-of-interest. Such binding domains can be synthesized as part of the linker or as distinct molecules which can be non-covalently associated with a core molecule to form the linker (linker complex).

Non-covalent association of binding domains to linkers can be advantageously used to enable the linkers of the present invention to be modular, such that one type of molecular linker core can be used to associate essentially any desired binding domain according to the target molecule to be complexed and crystallized.

Binding domains which bind molecules of a molecule-of-interest covalently or binding domains which bind molecules of a molecule-of-interest non-covalently can be used, depending on the application and purpose.

Binding domains which bind a molecule-of-interest non-covalently can be advantageously used to bind a molecule-of-interest without the need to resort to chemical synthesis techniques required for covalently coupling molecules. In the case of a biomolecular molecule-of-interest, the availability of highly specific ligands, such as, for example, antibodies, provides a pool of molecules useable as highly efficient binding domains.

Binding domains which bind a molecule-of-interest covalently can be advantageously used to bind a molecule-of-interest with great stability, thereby minimizing conformational disorder in crystals generated therewith, relative, for example, to binding domains which bind a molecule-of-interest non-covalently.

Preferably, single molecule linkers are porphyrin based. Porphyrin based linkers can be advantageously used to multimerize molecules of a molecule-of-interest with great stability and rigidity, as described in Example 1 of the following Examples section.

Multimerized streptavidin or streptavidin derived molecules may be advantageously utilized as the core of a molecular linker.

Preferably, the streptavidin molecule or streptavidin derived molecule is a core streptavidin. Suitable core streptavidins may comprise, for example, amino acid residues 13-133, 13-131 or 16-131 of native streptavidin.

The use of core streptavidin as the core of molecular linkers is advantageous since core streptavidin homomultimerizes into a particularly tightly packed tetramer, for example relative to native streptavidin tetramer. As a further advantage, core streptavidin tetramers display enhanced stability under denaturing conditions, and their biotin binding sites appear to be more accessible relative to native streptavidin tetramer. Extensive literature exists for the expression, purification and uses of streptavidin or streptavidin derived molecules (Wu S C. et al., 2002. Protein Expression and Purification 24:348-356; Gallizia A. et al., 1998. Protein Expression and Purification 14:192-196), fusion proteins comprising streptavidin or streptavidin derived molecules (Sano T. & Cantor C R. 2000. Methods Enzymol. 326:305-11), and modified streptavidin or streptavidin derived molecules (see, for example: Sano T. et al., 1993. Journal of Biological Chemistry 270:28204-28209), including for streptavidin or streptavidin derived molecules whose gene sequence has been optimized for expression in E. coli (Thompson L D. & Weber P C., 1993. Gene 136:243-6).

Fusion proteins comprising core streptavidins may be optimal when comprising an N-terminal core streptavidin segment and/or when produced as inclusion bodies. This may optimize correct folding and/or maximize the number of free biotin binding sites.

Molecular linkers including multimerized fusion proteins comprising core streptavidin and a polypeptidic binding domain, such as a single chain antibody Fv or a biological ligand of the molecule-of-interest, can be conveniently used to efficiently crystallize a molecule-of-interest.

Synthesis of chimeric polypeptides comprising core streptavidin and a single chain Fv can be effected by cloning nucleic acid sequences encoding the single chain Fv in an expression vector configured to express an in-frame chimeric polypeptide comprising core streptavidin, and the single chain Fv in a suitable host such as E. coli following transformation thereof using standard recombinant polypeptide expression technology.

Detailed protocols for the synthesis of streptavidin-single chain Fv fusion proteins can be found in the literature of the art (for example refer to Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

As is shown in Examples 7 and 9 of the Examples section which follows, core streptavidin based molecular linkers can be used to crystallize a molecule-of-interest.

Suitable binding domains which bind a molecule-of-interest non-covalently include but are not limited to, polypeptides derived from antibodies, such as, for example, single-chain Fv fragments, as described in Example 7 of the Examples section, T cell receptors, MHC-peptide complexes, biological ligands of the molecule-of-interest, and affinity-selected peptides, such as phage-display selected peptides.

As described in Example 7 of the Examples section, single-chain Fv fragments can be advantageously used to specifically bind and crystallize a molecule-of-interest.

In general, synthesis a single chain Fv molecule specific for a molecule-of-interest comprises producing and screening hybridoma cell lines secreting an antibody specific for the molecule-of-interest via standard hybridoma production techniques, and using RT-PCR to clone cDNA sequences encoding the variable light and variable heavy chains of the antibody. Ample guidance regarding production of single chain Fv's and fusion proteins comprising single chain Fv's is available in the literature of the art.

Suitable binding domains which bind a molecule-of-interest covalently include various chemical groups such as, for example, [—N⁺(CH₃)₃] and [—CO(CF₃)] (trifluorocarbonyl), as described in Example 1 of the Examples section, and N-(5-amino-1-carboxypentyl)imino-diacetic acid (NTA), as described in Example 11 of the following Examples section. Covalent coupling of a molecule-of-interest to a linker can be effected using standard chemical techniques for which guidance is broadly available in the literature of the art. For example, a trifluorocarbonyl group can be bound to the amino end, as well as to amino acid residues having free —OH, —SH, —NH2 groups of a polypeptidic molecule-of-interest, via a reaction of these groups with a compound such as HO—C(═O)—CF₃, under appropriate conditions.

It will be appreciated that other than as described hereinabove, linker universality can also be achieved by modifying the molecule to be crystallized to include specific binding moieties recognized by a single and universal linker, for example as described in Example 8 of the Examples section below. In the case of a polypeptidic molecule-of-interest, the molecule-of-interest can be expressed as part of a chimeric polypeptide including the binding moiety. Alternatively, the moiety is chemically attached to the molecule-of-interest. In any case, the binding moiety is preferably selected such that it readily associates with the linker while not substantially modifying the structure of the molecule to be crystallized.

Examples of binding domains of such universal linkers include biotin, as described in Examples 2 and 4 of the Examples section, an antibody-derived molecule, such as an anti purification tag single-chain Fv fragment, as described in Example 7 of the Examples section, a nickel ion, as described in Example 11 of the Examples section below, or essentially any specific ligand of a purification tag.

Examples of moieties which can be used to modify a molecule-of-interest such that it may be bound by universal linkers comprising specific ligands of purification tags include various purification tags.

As used herein, the term “purification tags” encompasses affinity tags.

Examples of purification tags include epitope tags, histidine tags, Strep-tags, single-chain Fv molecules, core streptavidin, streptavidin, and biotin.

Guidance regarding tagging molecules with histidine tags, and uses of such molecules is available in the literature of the art (for example, refer to: Sheibani N. 1999. Prep Biochem Biotechnol. 29:77).

Guidance regarding tagging molecules with Strep-tags, and uses of such molecules is available in the literature of the art (for example, refer to: Schmidt, T G M. and Skerra, A. Protein Eng. 1993, 6:109; Skerra A. & Schmidt T G M., 1999. Biomolecular Engineering 16:79-86).

Epitope tags can be comprised in a molecule-of-interest to enable complexation with linkers comprising single-chain Fv domains specific for such epitope tags.

Examples of epitope tags include an 11-mer Herpes simplex virus glycoprotein D peptide, and an 11-mer N-terminal bacteriophage t7 peptide, being commercially known as HSVTag and T7 Tag, respectively (Novagen, Madison, Wis., USA), and 10- or 9-amino acid c-myc or Hemophilus influenza hemagglutinin (HA) peptides, which are recognized by the variable regions of monoclonal antibodies 9E10 and 12Ca5, respectively.

Examples of moieties which can be used to modify molecules of interest such that these may be bound by a linker comprising biotin include streptavidin, core streptavidin and anti biotin single-chain antibody Fv.

Examples of moieties which can be used to modify molecules of interest such that these may be bound by a linker comprising streptavidin include Strep-tags, as described in Example 8 of the Examples section, or biotin.

Examples of moieties which can be used to modify molecules of interest such that these may be bound by a linker comprising a metal atom include, but are not limited to, histidine tags.

In the case of polypeptidic molecules-of-interest, polypeptide tags, such as, for example, histidine tags or Strep-tags, are particularly convenient since the molecule-of-interest and the tag can be co-expressed as a chimeric protein.

As mentioned hereinabove, the linkers of the present invention facilitate crystallization of molecules of interest by enabling the generation of a molecule-linker complex in which bound molecules are positioned in a defined spatial orientation. To allow such spatial positioning, the linker is selected of a size and geometric configuration which is capable of restricting the bound molecules to a predetermined orientation thus greatly facilitating 3D crystal formation.

Linker size and geometric configuration selection are also influenced by the need to maximize molecule-molecule interactions during or following complex formation. Such molecule-molecule interactions enhance the stability of the complex formed and thus further facilitate crystal formation therefrom.

It will be appreciated that linker length and spatial configuration selection is effected in accordance with the molecule to be crystallized. Such selection may be advantageously facilitated using computerized 3D modeling of the assembled crystallization complex. Such computerized 3D modeling is routinely effected by the ordinarily skilled practitioner using software available via the Internet/World Wide Web. Suitable software applications which may be used to generate 3D structure models of molecules include RIBBONS (Carson, M. (1997) Methods in Enzymology 277: 25), O (Jones, T A. et al. (1991) Acta Crystallogr A47:110), DINO (DINO: Visualizing Structural Biology (2001) http://www.dino3d.org); and QUANTA, CHARMM, INSIGHT, SYBYL, MACROMODE, ICM, MOLMOL, RASMOL and GRASP (reviewed in Kraulis, J. (1991) Appl Crystallogr. 24:946).

For example, in the case of membrane proteins, a core streptavidin-single-chain Fv linker (Example 7) can be used to tetramerize a membrane protein to form a non-planar geometric configuration. Such a non-planar geometric configuration would prevent the membrane protein from forming disordered aggregates or 2D crystals and would thus enable the generation of 3t) crystals therefrom.

In the case of molecules which lack sufficient conformational rigidity, the linkers employed are designed so as to provide rigidity to bound molecules thereby further facilitating crystallization thereof.

Such conformational rigidity can be obtained by utilizing linkers having cores based on polydentate ligands, including, but not limited to, polydentate ligands, such as porphyrin, or macrobicyclic cryptands, such as hydroxime, as described in Examples 1-5 and 11 of the Examples section which follows. As described hereinabove, core streptavidin tetramer can be used to generate a suitably conformationally rigid linker.

In addition to the above described features, the linkers employed by the present invention can also include several additional features.

According to another preferred embodiment of the present invention, the linkers include a hydrophilic domain such that complexes formed thereby are sufficiently hydrophilic so as to facilitate crystallization of molecules of interest which are substantially hydrophobic.

Examples of such “hydrophilic” linkers include, for example, linkers comprising core streptavidin or single-chain Fv, as described in Example 7 of the Examples section, linkers comprising non-polypeptidic hydrophilic molecules such as, for example, trimethylammonium, as described in Example 5 of the Examples section, or linkers comprising N-(5-amino-1-carboxypentyl)imino-diacetic acid (NTA) groups, as described in Example 11 of the Examples section below.

According to another preferred embodiment of the present invention, the linkers include a purification tag, for example, as described hereinabove. Such a purification tag can be advantageously used for purification of the linker and/or of the molecular complex.

Purification of a molecule-of-interest is a critical and limiting step in the crystallization of a molecule-of-interest, such as a polypeptidic molecule-of-interest and, as such, methods for improving such purification can serve to thereby greatly facilitate the crystallization of such molecules of interest. The same considerations may be applicable to purification of the linkers, such as the polypeptide-based linkers of the present invention.

Examples of suitable purification tags include, for example, the epitope tags to which specific antibodies exist which are listed and described hereinabove, a Strep-tag and a histidine tag, as described in Example 7 of the Examples section. Purification of a molecule containing a histidine tag is routinely performed by those well-versed in the art, using nickel-based automatic affinity column purification techniques. Purification of a molecule containing a Strep-tag can be effected using standardized techniques, for example, as described hereinabove.

The method of the present invention can be used to crystallize any known type of molecules including inorganic and organic molecules.

Examples of organic molecules include, but are not limited to, polypeptides such as membrane proteins, receptors, enzymes, antibodies and prions, as well as nucleic acids, carbohydrates, hormones, polycyclic molecules and lipids.

The present invention can be advantageously used to crystallize a GPCR.

Preferably, the present invention is used to crystallize a GPCR such as rhodopsin or a class A GPCR.

Preferably, the present invention is used to crystallize a class A GPCR such as m2 muscarinic cholinergic receptor.

Guidance regarding families, types or classes of GPCRs, including mutant GPCRs, is widely available in the literature of the art (see, for example: Edvardsen O. et al., 2002. Nucleic Acids Res. 30:361; Attwood T K. et al., 2002. Protein Eng. 15(1):7)

Crystallization of GPCRs is preferably effected using molecular linkers comprising as a binding domain a GPCR-binding domain of an arrestin molecule.

Types of arrestins which can be used according to the method of the present invention include, but are not limited to, beta-arrestin-1a (Lohse M J. et al., 1990. Science 248:1547-1550; Parruti, G. et al., 1993. J Biol Chem. 268:9753-9761; Calabrese G. et al., 1994. Genomics 24:169-171; Lefkowitz R J., 1998. J Biol Chem. 273:18677-18680; Luttrell L M. et al., 1999. Science 283:655-661), arrestin-C (Craft C M. et al., 1994. J Biol Chem. 269:4613-4619), S-arrestin (Yamaki K. et al., 1990. J Biol Chem. 265:20757-20762; Calabrese G. et al., 1994. Genomics 23:286-288; Yamamoto S. et al., 1997. Nat Genet. 15:175-178; Sippel K C. et al., 1998. Invest Ophthalmol Vis Sci. 39:665-670), arrestin 3 (Murakami A. et al., 1993. FEBS Lett. 334:203-209; Craft C M. et al., 1994. J Biol Chem. 269:4613-4619; Sakuma H. et al., 1996. FEBS Lett. 382:105-110), beta-arrestin-2 (Rapoport B. et al., 1992. Mol Cell Endocrinol. 84:R39-R43; Attramadal H. et al., 1992. J Biol Chem. 267:17882-17890; Calabrese G. et al., 1994. Genomics 23:286-288; Lefkowitz R J., 1998. J Biol Chem. 273:18677-18680), and beta-arrestin-1b (Lohse M J. et al., 1990. Science 248:1547-1550; Parruti G. et al., 1993. J Biol Chem. 268:9753-9761; Calabrese G. et al., 1994. Genomics 24:169-171; Lefkowitz R J., 1998. J Biol Chem. 273:18677-18680; Luttrell L M. et al., 1999. Science 283:655-661). Ample guidance regarding the location of G protein coupled receptor binding domains of arrestins is provided in the aforementioned references and in the Examples section which follows.

Preferably, the arrestin molecule is beta-arrestin-1a.

Regardless of the type of arrestin used, the GPCR binding domain is preferably homologous to amino acid residues 11 to 190, or 11 to 370 of human beta-arrestin-1a.

Preferably, the G protein coupled receptor-binding domain has a mutation at an amino acid residue position corresponding to position 90 in bovine visual arrestin, a mutation at an amino acid residue position corresponding to position 175 in bovine visual arrestin, or more preferably both.

Preferably, the mutation at an amino acid residue position corresponding to position 90 in bovine visual arrestin is a mutation to a threonine residue or more preferably to a serine residue.

Preferably, the mutation at an amino acid residue position corresponding to position 175 in bovine visual arrestin is a mutation to a an asparagine residue or more preferably to a glutamic acid residue.

Guidance regarding identification of amino acid residue positions in various arrestins corresponding to amino acid residue positions in bovine visual arrestin can be found in the literature of the art (see, for example: Han M. et al., 2001. Structure (Camb) 9:869-80; Hirsch J A. et al., 1999. Cell 97:257-69).

In general, corresponding amino acid residue positions between any pair of related proteins, such as a pair of arresting, may be computationally determined using software tools suitable for aligning proteins, such as alignment software of the NCBI available on the World Wide Web/Internet.

As is described in Example 9 of the following Examples section, GPCR-binding domains of arrestins having a serine residue at an amino acid residue position corresponding to position 90, or a glutamic acid residue an amino acid residue position corresponding to position 175 in bovine visual arrestin can, respectively, be advantageously used to bind different types of GPCRs or to bind GPCR independently of its activation-phosphorylation state, respectively.

Preferably, the GPCR binding domain corresponds to the amino acid sequence set forth in SEQ ID NO: 3 or SEQ ID NO: 4. As shown in Example 9 of the Examples section below molecular linkers comprising SEQ ID NO: 3 or SEQ ID NO: 4 can be used to specifically bind various types of GPCRs with high affinity and specificity regardless of the activation state of such GPCRs.

Crystallization of the linker-molecule complex can be effected via any of the standard means described in the literature, including, for example, microbatch, vapor diffusion or dialysis (Bergfors, T. M., Protein crystallization. IUL Biotechnology Series. 1999, La Jolla, Calif.: International University Line). In such methods, the appropriate amount of linker is added to a monodisperse solution of the molecule-of-interest and the solution is then employed in any of the methods mentioned above. For example, the optimal amount of reagents, such as linker subunits, to be added for facilitating crystallization can be determined by dynamic light scattering so as to ensure monodispersity of the crystallizable molecular complex and to measure the second virial coefficient, which can be employed as a diagnostic indicator for the tendency of the molecular species in solution to crystallize (George, A., et al., Macromolecular Crystallography, Pt a. 1997. p. 100).

To facilitate X-ray crystallographic determination of the structure of a crystallized molecule-of-interest, the molecular complexes of the present invention can further include at least one metal atom associated therewith. Such a metal atom can be used to generate initial phases for X-ray diffraction crystallography, via methodologies such as multiple anomalous diffraction (MAD) (Hendrickson W A., Science 1991, 254:51), thereby facilitating solution, for example, of the 3D atomic structure of the crystallized molecule.

Alternately, X-ray crystallographic structure determination of the molecule-of-interest may be facilitated by association of a metal atom with the molecule-of-interest.

Examples of such metal atoms include, for example, iron, cobalt, nickel, cadmium, platinum and zinc.

To be capable of associating with a metal atom, the linkers of the present invention may include polydentate ligands, such as porphyrin, and macrobicyclic cryptands, such as hydroxime, as described in Example 1 of the Examples section.

Alternately, to be capable of associating with a metal atom, the linkers of the present invention or a molecule-of-interest may include, for example, a metal binding protein, such as metallothionein, desulforedoxin, rubredoxin, colicin or rubrerythrin.

Preferably, the metal binding protein is metallothionein.

Conjugation of a metal binding protein with a polypeptidic linker or molecule-of-interest can be conveniently effected by co-expressing the metal binding protein with the linker or the molecule-of-interest as a fusion protein.

For example, metallothionein-streptavidin fusion proteins may be generated as previously described (Sano T. et al., 1999. Proc Natl Acad Sci USA. 89:1534-8).

As shown in Example 9 of the Examples section below, a molecular linker comprising metallothionein can be used to generate a highly ordered crystal of a membrane protein, which crystal comprising a metal atom useful for determining initial phases for structural analysis of such a membrane protein.

It will be understood by one versed in the art that metal atoms facilitating crystallographic analysis, as described in the present invention, include the ionized forms of such metal atoms, such as, for example, Pt²⁺, Ni²⁺, Cu²⁺ or Co²⁺.

It will be appreciated that such a metal atom can also serve as a nucleating core around which linker arms can associate into a linker complex as described hereinabove.

Thus, the present invention enables crystallization of any molecule-of-interest and, in particular, hydrophobic and amphiphilic molecules which are difficult or impossible to crystallize using prior art methods.

In sharp contrast to the linkers used by prior art methods, the linker configurations used by the method of the present invention:

(i) are capable of forming molecular complexes with molecules of interest of a sufficient solubility so as to facilitate crystallization thereof,

(ii) can be easily modified to include binding moieties specific for virtually any region of any molecule-of-interest,

(iii) are designed so as to direct the spatial positioning and/or orientation of bound molecules thereby facilitating crystallization thereof, and

(iv) are designed so as to provide structural rigidity to bound molecules thereby facilitating crystallization thereof.

Aside from enabling crystallization and subsequent atomic structure determination of previously uncharacterized molecules, the capacity of the present invention to multimerize and/or purify a molecule-of-interest can be advantageously applied in various biomedical fields including protein therapeutics, oral lumenal therapies for gastrointestinal diseases and self-adjuvanting or subunit vaccines.

In addition, crystallization of macromolecule pharmaceuticals, and in particular proteins, can be used to streamline manufacturing processes, as in the case with small-molecule drugs. Since a crystal is the most concentrated possible form of a protein, crystallization can be beneficial for drugs, such as antibodies, which require high doses at the delivery site. In addition, since the rate of crystal dissolution depends on its morphology, size, and the presence of excipients, crystalline proteins may also serve as a convenient carrier-free slow release dosage form (insulin is a good example). Finally, the stability of proteins in crystalline form is higher than that of corresponding soluble or amorphous materials and, as such, crystallization can be used to greatly increase the shelf life of a drug product.

Macromolecular crystals generated according to the teachings of the present invention also find important uses as catalysts, adsorbents, biosensors and chiral chromatographic media. These may also be employed in environmental applications, including, for example, the destruction of nerve agents, for bioremediation and civil defense.

In addition to the above, the present invention provides methods of protein purification via crystal formation.

As described hereinabove, suitable GPCR-binding domains of arrestin molecules can be used to bind GPCRs with high affinity and specify. Such GPCR binding domains of arrestin molecules can therefore be used as affinity ligands for purification of such GPCRs.

Thus, according to the present invention, there is provided a method of purifying a GPCR from a sample containing a GPCR.

The method of purifying a GPCR from a sample is effected by subjecting the sample to affinity chromatography using a GPCR binding domain of an arrestin molecule.

All criteria described hereinabove regarding selection and/or modification of a GPCR binding domain of an arrestin molecule suitable as a binding domain of a molecular linker are applicable to selection and/or modification of a GPCR binding domain of an arrestin molecule suitable as a GPCR binding region of an affinity ligand for the presently described purification method. As is described in Example 10 of the Examples section below GPCR binding domains of an arrestin molecule corresponding to SEQ ID NO: 3 or SEQ ID NO: 4 can be used to efficiently bind various types of GPCRs with high specifity and affinity, and thereby to efficiently purify various GPCRs regardless of the activation-phosphorylation state thereof.

Preferably the affinity ligand includes a purification tag for facilitating attachment of the affinity ligand to an affinity chromatography matrix.

As is described in Example 10 of the Examples section below an affinity ligand conjugated to a Strep-tag can be conveniently bound to an affinity matrix to which core streptavidin is conjugated.

Alternately, as is further described in Example 10 of the Examples section below an affinity ligand conjugated to core streptavidin can be conveniently bound to an affinity matrix to which a Strep-tag or iminobiotin is conjugated.

Suitable protocols for all phases of affinity chromatography purification of molecules are widely available in the literature of the art (see, for example: Wilchek M. & Chaiken I., 2000. Methods Mol Biol 147:1-6; Jack, G. W. Immunoaffinity chromatography. Mol Biotechnol 1, 59-86; Narayanan S R., 1994. Journal of Chromatography A 658:237-258; Nisnevitch M. & Firer M A., 2001. J Biochem Biophys Methods 49:467-80; Janson J C. & Kristiansen T. in Packings and Stationary Phases in Chromatography Techniques (ed. Unger, K. K.) 747 (Marcel Dekker, New York, 1990); Clonis, Y. D. in HPLC of Macromolecules A Practical Approach 157 (IRL Press, Oxford, 1989); Nilsson J. et al., 1997. Protein Expr Purif. 11:1-16).

Preferably, the present invention is used to purify a GPCR such as rhodopsin or a class A GPCR.

Preferably, the present invention is used to purify a class A GPCR such as m2 muscarinic cholinergic receptor.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Generation of Ordered Crystals of Molecules of Interest by Complexation Thereof with Non-Polypeptidic Molecular Linkers

In order to facilitate ordered crystallization and atomic structure determination of a molecule-of-interest, non-polypeptidic molecular linkers were designed having the capacity to form a crystallizable molecular complex with molecules of a molecule-of-interest and, preferably, with a metal atom.

Materials and Methods:

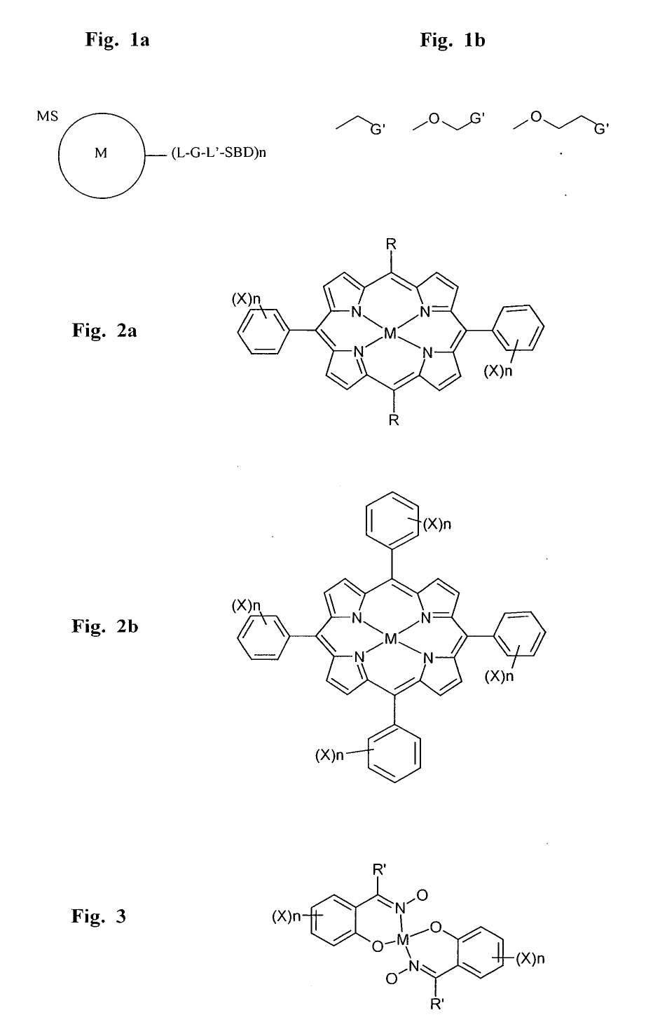

Molecular linkers are generated to facilitate ordered crystallization of molecules-of-interest having the following characteristics: (a) the ability to homomultimerize molecules-of-interest in selected geometric configurations, thereby facilitating ordered crystallization of molecules-of-interest which do not naturally aggregate in configurations suitable therefor; (b) sufficient conformational rigidity so as to facilitate ordered crystallization or ordered assembly of molecules-of-interest lacking sufficient conformational rigidity therefor; (c) sufficient hydrophilicity so as to facilitate solubilization in polar solvents, and thereby crystallization, under standard crystallization-inducing conditions of molecules-of-interest lacking sufficient hydrophilicity therefor, (d) binding moieties specific for desired regions of molecules-of-interest, thereby facilitating multimerization of the molecules-of-interest; and (e) the ability to specifically bind a metal atom being capable of facilitating 3D crystallographic analysis of molecules-of-interest by enabling generation of initial phases for X-ray diffraction crystallography. A modular organization of such molecular linkers is schematized in FIG. 1a.

Such linkers may extend a binding moiety from a multimerization scaffold via a first chain of 1-3 carbon or oxygen atoms, representative examples of which are depicted in FIG. 1b. These chains preferably terminate in a functional group such as [—CO₂H], [—OH], [—NH₂], [—CO₂], [—CONH], [—O], [—OCO] or [—NHCO] which are used to attach, via conventional ester, amide or ether formation, a second chain of suitable length and geometry so as to enable attachment of monomers of a molecule-of-interest to the multimerizing scaffold of the molecular linker in the desired spatial configuration. Such chains preferably include a molecular group, such as [—(CH₂CH₂O)₂—O—CH₂CH₂—] or [—(CH₂)₄—], to which is attached the binding moiety. Such chains possess sufficient conformational rigidity and/or hydrophilicity so as to facilitate crystallization of molecules of a molecule-of-interest complexed therewith lacking such conformational rigidity and/or hydrophilicity, respectively.

Moieties specific for binding molecules of interest are preferably polypeptides capable of directly or indirectly mediating specific recognition of a molecule-of-interest, such as core streptavidin, peptide tags or antibodies. Alternatively, molecules such as [—N⁺(CH₃)₃] or [—CO(CF₃)] can be employed to specifically bind a molecule-of-interest. Binding of metal atoms to molecular linkers can be effected via the use of molecular linkers comprising multimerization scaffolds based on molecules, such as porphyrin or hydroxime, which can bind metal atoms such as Pt²⁺, Ni²⁺, Cu²⁺ or Co²⁺.

Examples of molecular linkers capable of forming a crystallizable molecular complex with a molecule-of-interest and specifically binding a metal atom include, for example, porphyrin-based molecular linkers (FIGS. 2a and 2b, respectively) or hydroxime-based molecular linkers (FIG. 3).

Thus, the molecular linkers of the present invention form molecular complexes with molecules of a molecule-of-interest being positioned in a selected spatial geometry facilitating crystallization thereof. Such molecular linkers further facilitate crystallographic analysis of a molecule-of-interest by incorporating within the crystallizable molecular complex a metal atom used to generate initial phases during X-ray crystallography.

### Example 2

### Chemical Synthesis of Porphyrin-Based Molecular Linkers

As described in Example 1, porphyrin-based molecular linkers can be employed to facilitate crystallization of molecules of interest by multimerizing these within substantially conformationally rigid and/or hydrophobic crystallizable molecular complexes. Such linkers further facilitate determination of the atomic structure of molecules of interest by incorporating a platinum atom which can be employed to generate initial phases during X-ray crystallographic analysis of crystals of such molecular complexes.

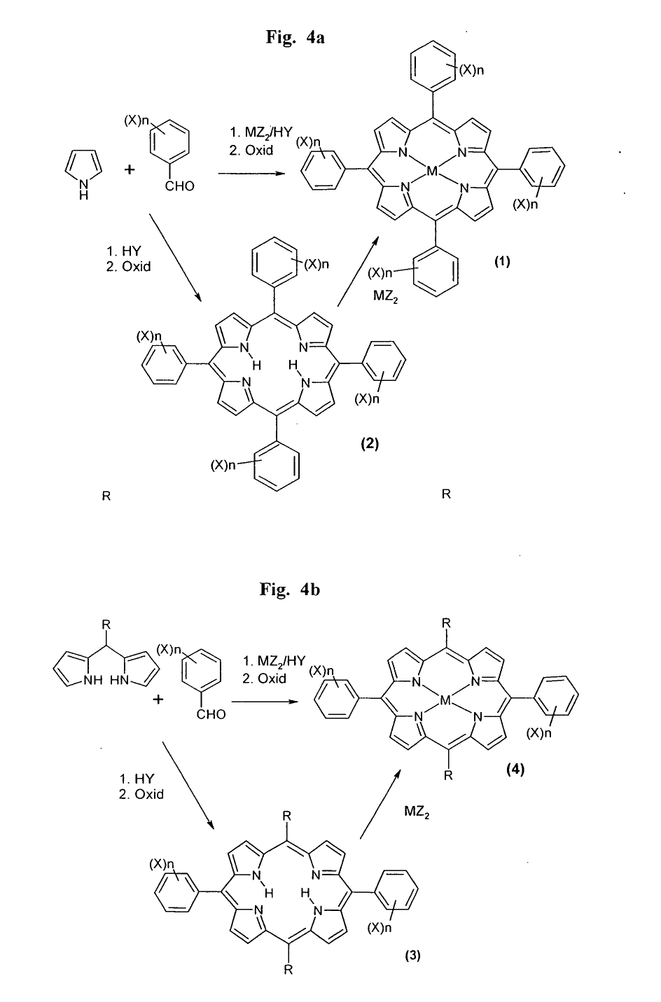

Synthetic procedures to generate crystallizable molecular complexes with porphyrin-based molecular linkers are depicted in FIGS. 4a and 4b.

The steps involved in synthetic processes to generate a porphyrin-based molecular linker, 5,15-Di(2,6-di(ethoxycarbonymethoxy))porphyrinato-platinum (FIG. 4b, Product No. 4), and the attachment of various molecular spacers/binding domains thereto are outlined below:

Materials and Methods:

Synthesis of 5,15-Di(2,6-di(ethoxycarbonymethoxy))porphyrin (Product No. 3): Dipyrrolmethane (280 mg, 1.9 mmol) and 2,6-di(ethoxycarbonymethoxy)-benzaldehyde (590 mg, 1.9 mmol) were dissolved in dichloromethane (300 ml) and purged with nitrogen. To this was added trifluoroacetic acid (75 ml, 1 mmol) and the solution was stirred for 3 hours at room temperature. DDQ (450 mg, 2 mmol) was added, the mixture was stirred for 1 hour and neutralized with triethylamine (1.5 ml). The resultant mixture was purified by chromatography on a silica column, eluting with dichloromethane. The product was eluted as a purple band from the column and was obtained by evaporation of the eluate to give purple crystals (250 mg) of the product.

Synthesis of 5,15-Di(2,6-di(ethoxycarbonymethoxy))-porphyrinatoplatinum (Product No. 4, where X=OCH₂CO₂Et, R=H, n=2M=Pt): The product of the previous reaction (250 mg) was dissolved in acetic acid (50 ml) and to this was added dipotassium tetrachloroplatinate (112 mg) and the mixture was refluxed for 10 min. The mixture was cooled and water (20 ml) was added. The product (350 mg) was filtered off and washed with 50% aqueous ethanol.

Synthesis of 5,15-Di(2,6-di(ethoxycarbonymethoxy))porphyrinatoplatinum (Product No. 4, where X=OCH₂CO₂H, R=H, n=2, M=Pt): The product of the previous step (350 mg) was suspended in 50% aqueous ethanol (50 ml) containing sodium hydroxide (500 mg) and refluxed for 3 hrs. The mixture was then acidified drop-wise with concentrated HCl, to pH 1 and the product (280 mg) was filtered off.

Synthesis of 5,15-Di(2,6-di((N-biotinylaminopropyl)amidocarbonymethoxy))porphyrinatoplatinum (Product No. 4, where X=OCH₂CO₂NH—(CH₂)₃NH(biotinyl), R=H, n=2, M=Pt): 350 mg, 288 mmol of the product of the previous reaction was added to a solution of DCC (72 mg) in dioxane (100 ml) containing a catalytic amount of hydroxybenzotriazole (5 mg). 3-(biotinylamino)-propylamine (95 mg, 320 mmol) was then added and the mixture was stirred overnight at room temperature and filtered. The residue was washed with ethyl acetate and the filtrate was evaporated to give the crude product (605 mg). The product was then be further purified by chromatography on a silica gel column, eluting with ethyl acetate. Analogues of this compound are synthesized similarly.

The number of moieties specific for the molecule-of-interest are given by the index n. The steric encumbrance between such moieties determine the geometry of the molecular scaffold, and thus the geometry of the molecule-of-interest-linker complex. The biotinyl moiety described above can be used, for example to bind any molecule-of-interest which has been fused to streptavidin.

### Example 3

### Chemical Synthesis of a Hydroxime-Based Molecular Linker

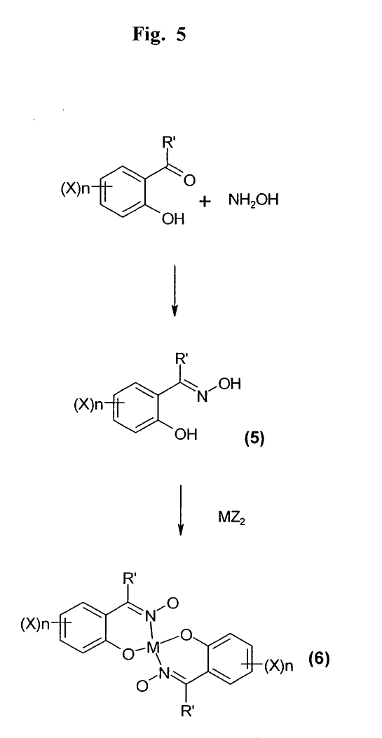

A synthetic procedure to generate a hydroxime-based molecular linker for binding two molecules of a molecule-of-interest, thereby generating a crystallizable molecular complex containing the molecule-of-interest, is depicted in FIG. 5. Such a molecular linker further facilitates determination of the crystal structure of the molecule-of-interest by chelating a copper atom which is employed to generate initial phases during X-ray crystallographic analysis of a crystal of the molecular complex.

Materials and Methods:

Synthesis of 5-((2-trimethylammonium-ethoxy)digolyloxycarbonyl)-2-hydroxyacetophenone oxime dichloride (intermediate No. 5 where X=CO.(OCH₂CH₂)₃N⁺Me₃, R′=Me, n=1): 5-Carboxy-2-hydroxyacetophenone oxime (1 g, 6 mmol) was dissolved in dioxane (50 ml) containing DCC (0.95 g) and (2-trimethylammonium-ethoxy)-digol chloride (1.4 g) dissolved in dioxane (20 ml) and the mixture was stirred for 6 hours at room temperature. The mixture was filtered and the filtrate was evaporated to dryness. The residue was then dissolved in water and the product was purified by ion exchange chromatography on a Dowex cation exchange column and was obtained as a viscous oil, on evaporation under high vacuum, as a chloride salt.

Synthesis of Bis-[5((2-trimethylammonium-ethoxy)-digolyloxycarbonyl)-carboxy-2-hydroxyacetophenone oxime] copper (II) chelate dichloride (Product No. 6, where X=CO.(OCH₂CH₂)₃N⁺Me₃, R′=Me, n=1, M=Cu): 100 mg of the previous reaction product was dissolved in water (10 ml) and to this was added an aqueous solution of copper (II) chloride (1.5 ml of 0.1M solution). The solution was stirred for 4 hours and the mixture was evaporated to dryness, under high vacuum, to yield the product (110 mg) as a green solid. Analogues of this compound are synthesized similarly.

The quaternary ammonium moiety is employed to bind any molecule which is known to bind positively charged groups via cation-π interactions, such as acetylcholinesterase.

### Example 4

### Synthesis of a Non-Polypeptidic Molecular Linker with Biotinylated Moieties for Attachment of a Molecule-of-Interest Coupled to a Biotin-Binding Molecule

A modular system where a single type of molecular linker may bind a range of molecules of interest is highly desirable since this obviates the requirement of synthesizing a dedicated linker for each molecule-of-interest. This is effected, for polypeptides of interest, for example, by incorporating within the molecular linker and the polypeptide of interest heterologous moieties, such as polypeptides, that specifically bind to each other.

Since one of the highest binding affinities known between any two non-covalently associated molecules is that between core streptavidin and biotin, the use of such binding a pair is ideal for binding a molecule-of-interest to a molecular linker. Such a binding interaction serves to optimize crystallization of the molecule-of-interest since it facilitates formation of a highly stable and rigid molecular complex which can be easily crystallized.

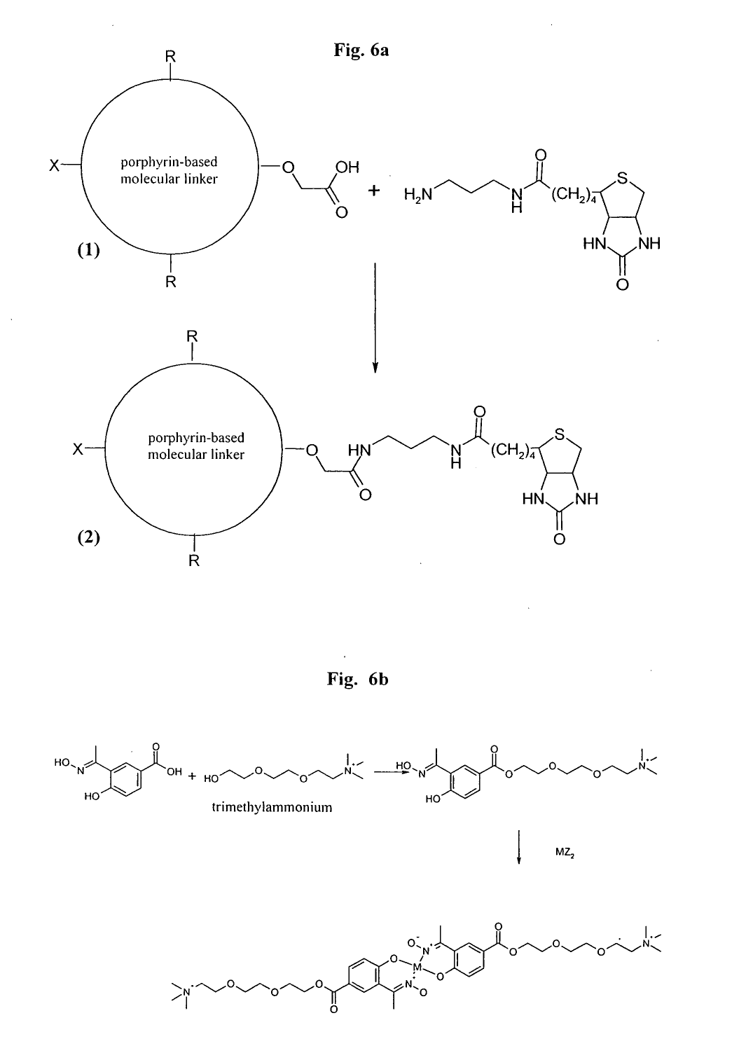

The synthetic process for linkage of a biotin moiety to a porphyrin-based molecular linker is outlined in FIG. 6a and is performed as follows:

Synthesis of 5,10,15,20-tetra-(3-ethoxycarbonyl)porphyrin (Product No. 2), where X=OCH₂CO₂Et, n=1): Ethyl 3-formylbenzoate (5 g) and pyrrole (2 g) were dissolved in chloroform (1 liter) and the solution was purged with nitrogen for 10 min. A solution of BF₃.Et₂O (4 ml of 2.5M solution). After 1 hour chloranil (5.4 g) was added and the mixture was refluxed for 1 hour. The mixture was cooled to room temperature and 1 equivalent of triethylamine was added. The solution was evaporated to dryness to give the crude product, which was washed with methanol three times. The product remained as a purple solid (1.43 g). The product was then elaborated, analogously to the method described above for synthesis of porphyrin-based molecular linkers, into further examples of the invention.

### Example 5

### Synthesis of a Hydroxime-Based Molecular Linker with Trimethylammonium Moieties for Attachment of Molecules of a Molecule-of-Interest

In order to bind molecules of a molecule-of-interest in the desired spatial configuration within a crystallizable molecular complex a molecular linker, according to the method of the present invention, must be of a suitable dimension and geometry.

Such positioning of a molecule-of-interest within a crystallizable molecular complex is effected, for example, by employing molecular linkers with a hydroxime-based multimerization scaffold, as described above, to which molecules of a molecule-of-interest are attached via trimethylammonium moieties. As well as allowing binding of molecules of interest to a molecular linker without steric hindrance, trimethylammonium, being of substantial hydrophilicity and conformational rigidity, further facilitates solubilization and crystallization, respectively, of the molecular complex.

The chemical attachment of trimethylammonium to a hydroxime-based molecular linker is depicted in FIG. 6b. As described above, inclusion of a metal atom within the hydroxime-based molecular linker facilitates determination of the atomic structure of the molecule-of-interest by providing initial phases during X-ray crystallographic analysis of a crystal of a molecular complex including a molecule-of-interest.

### Example 6

### Crystallizable Molecular Complexes Comprising a Mutagenesis Polypeptide of Interest and a Heterologous Molecular Linker

Mutagenesis of a polypeptide of interest is employed so as to optimize the crystallizability of a molecular complex formed by a linker therewith.

The polypeptide of interest is mutagenized in order to adjust the steric fit between the molecular linker and the molecules of the polypeptide of interest. Such an adjustment is employed in order to optimize the number and/or physico-chemical characteristics of the crystal contacts of the crystallizable molecular complex formed by association of molecules of the polypeptide of interest with the molecular linker. Additionally, selected residues of the polypeptide of interest are mutagenized in order to optimize the solubility and/or rigidity of the crystallizable molecular complex formed by association of molecules of the polypeptide of interest with the molecular linker.

Acetylcholinesterase (AChE) and muscarinic acetylcholine receptor (mAChR) are molecules which are well characterized pharmacologically and AChE is known to crystallize in a series of well-characterized lattices. Thus, AChE is mutagenized so as to optimize its packing within a molecular linker when multimerized therewith.

Muscarinic acetylcholine receptor, whose 3D structure remains to be determined, is representative of a broad class of integral membrane proteins of great pharmacological importance. However, it is known to bind ligands possessing a similar structure to those binding AChE. Thus a modified molecular linker, based on the one employed for crystallization of mutagenized AChE, as described above, is employed in order to crystallize mAChR, an integral membrane protein.

Materials and Methods:

The molecule-of-interest is mutagenized via standard recombinant techniques and is produced using a bacterial expression system. The purified protein is solubilized in a monodisperse solution according to standard crystallization procedures available in the literature. To this solution, a suitable amount of molecular linker is added. A 5 microliter aliquot of this molecular linker solution is added to 5 microliters of mother solution on a siliconized glass coverslip (18-22 mm diameter). The coverslip is placed over a well containing a solution buffered at the appropriate pH and adjusted to the optimal concentration of precipitants (e.g. PEG 5000 or ammonium sulfate). The drop is allowed to equilibrate at the appropriate temperature (e.g. 20° C.) for an amount of time necessary for the crystal to form.

### Example 7

### Crystallization of a Molecule-of-Interest by Complexation with a Molecular Linker Composed of a Homomultimerizing Molecule Conjugated to a Modular Recognition Domain Specific for a Molecule-of-Interest

One of the most versatile, convenient and specific means of specifically binding a molecule-of-interest is via antibodies.

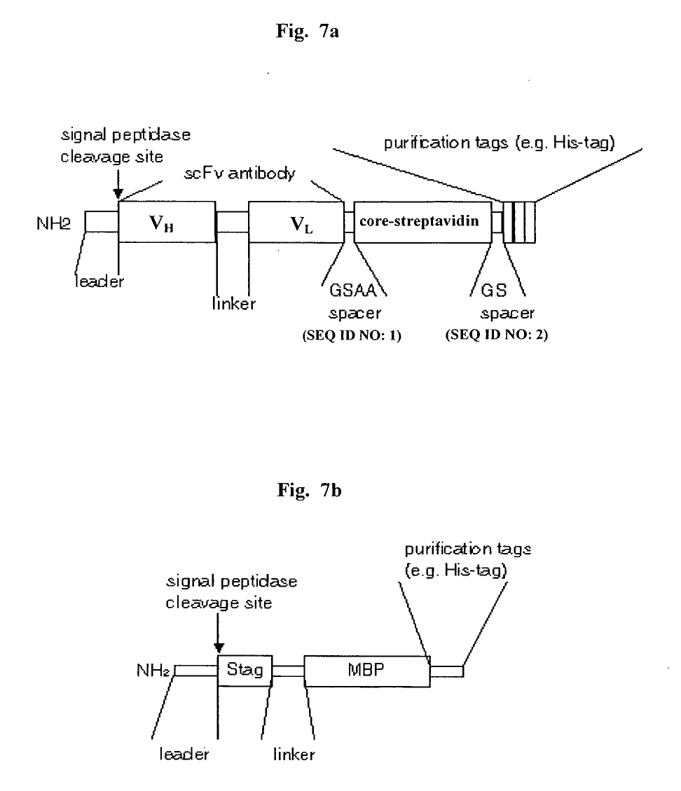

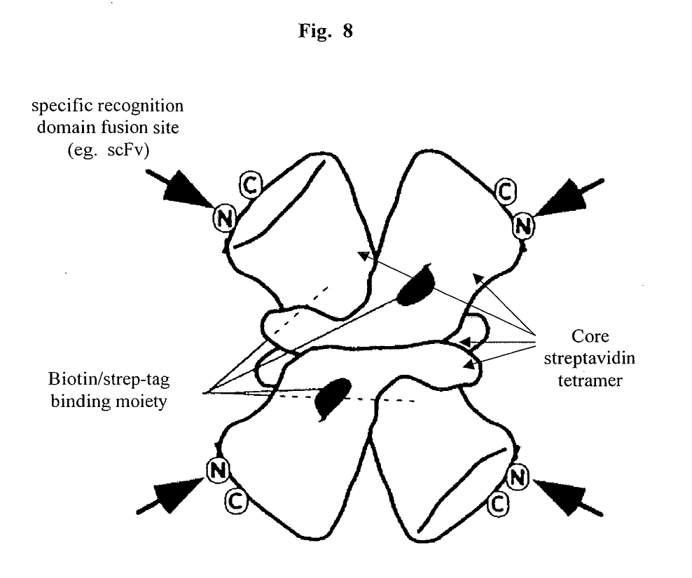

Therefore, molecular linkers were designed consisting of a chimeric polypeptide composed of fused scFv, core streptavidin and histidine tag segments, as depicted schematically in FIG. 7a. Such single-chain Fv-core streptavidin chimeric polypeptides and polypeptides including histidine tags have been previously described (Ladner, R. C. et al., U.S. Pat. No. 4,946,778) and (Sheibani N., 1999. Prep Biochem Biotechnol. 29(1):77), respectively. The relative positions of the single-chain Fv molecule and the core streptavidin segments can also be inverted. The peptide sequences GSAA (SEQ ID NO: 1) and GS (SEQ ID NO: 2) are inserted between the V_{L} and core streptavidin, and between the core streptavidin and the His-tag domains, respectively, so as to provide the required flexibility for appropriate folding of the fusion protein.

Optionally, association of a metal atom with the crystallizable molecular complex is effected via the use of a second chimeric polypeptide comprising Strep-tag, metal atom-binding and purification tag segments, as depicted in FIG. 7b. The Strep-tag domain of this chimera serves to bind the core streptavidin domain of the core streptavidin-containing chimera described hereinabove and thus serves to associate the molecule-of-interest with a metal atom binding molecule. Binding of the metal atom to the metal atom binding domain is effected either prior to, concomitantly or following the binding steps described above. Furthermore, the purification tag of the metal atom binding chimera can be employed to perform the same functions as the purification tag comprised in the core streptavidin-containing chimera described above. The conformation of a tetramerized complex obtained using the above-described system is depicted in FIG. 8.

Such a molecular linker thus binds a molecule-of-interest via its scFv domain, tetramerizes via its core streptavidin domain and can be easily identified by immunoblotting analysis or purified by affinity chromatography, either prior to or following binding of a molecule-of-interest, via its purification tag domain.

One advantage of utilizing streptavidin as the core of molecular linkers, is that extensive literature exists for the expression and purification of streptavidin itself (Wu S C. et al., 2002. Protein Expression and Purification 24:348-356; Gallizia A. et al., 1998. Protein Expression and Purification 14:192-196) and of streptavidin fusion proteins (Sano T. & Cantor C R. 2000. Methods Enzymol. 326:305-11). Smaller and more stable streptavidins than the native form have been produced recombinantly (Sano T. et al., 1993. Journal of Biological Chemistry 270:28204-28209) and the gene sequence has been optimized for expression in E. coli (Thompson L D. & Weber P C., 1993. Gene 136:243-6). The tetramer of these smaller “cores” displays enhanced stability under denaturing conditions, and their biotin binding sites appear to be more accessible. A small core size is also preferable, as it helps to keep the size of the final polypeptidic molecular linker to a minimum, making the scaffold easier and cheaper to produce and purify. Smaller molecular linkers may be advantageous since, as a rule of thumb, a smaller and tightly packed multimerization scaffold will introduce less disorder in the final crystallization complex, thus ensuring optimal ordering of crystals.

Crystallization of a molecule-of-interest using the above-described molecular linkers is achieved as follows:

The chimeric polypeptide described above is produced in a first step via standard recombinant DNA, protein expression and protein purification techniques. In a second step, the molecule-of-interest is crystallized within a crystallizable molecular complex formed by tetramerization of the chimera via core streptavidin, thereby generating a molecular linker, and by binding of molecules of the molecule-of-interest to the scFv domains of the molecular linker.

The order in which these various non-covalent binding steps are effected can be essentially shuffled at will since these involve biological interactions occurring under similar physiological conditions. As discussed above, incorporation of a metal atom into a molecular complex containing a molecule-of-interest serves to facilitate solution of the 3D atomic structure of the molecule-of-interest.

The scheme outlined hereinabove for crystallization of a molecule-of-interest is highly modular and flexible and the components thereof are interchangeable while retaining the basic functionalities required for formation of a crystallizable molecular complex. For example, the molecule-of-interest-specific scFv domain is exchangeable with any other molecule specifically binding the molecule-of-interest. One such example is a toxin specific for a membrane receptor, as described in the embodiments of the present invention. This is effected by employing the genetic sequence encoding the toxin instead of that of the scFv during the recombinant DNA manipulation phase of this crystallization method. Similarly, the metal atom binding segment of the chimera described above is exchangeable, via chemical synthesis, with a non-polypeptidic metal chelating molecule, such as porphyrin or hydroxime described in Examples 4 and 5, respectively. When employing appropriate combinations of auxiliary functional domains within the molecular linker, the core streptavidin domain segment of the molecular linker is exchangeable with any other suitable homomultimerizing molecule.

An alternative method for association of a metal atom with the crystallizable molecular complexes of the present invention involves the use of a molecular linker composed of a single type of molecule which includes the metal atom binding segment as well as the molecule-of-interest-binding, homomultimerizing and purification tag segments. This is effected, for example, via a chimeric polypeptide including all these functional segments.

Thus, such molecular linkers can be employed to facilitate crystallization and 3D atomic structure determination of a molecule which can be bound by an antibody.

### Example 8

### Generation of Ordered Crystals of a Polypeptidic Molecule-of-Interest Via Expression as a Fusion Chimera with a Heterologous Homomultimerization Domain

In order to crystallize a polypeptidic molecule-of interest, the molecule-of-interest is expressed as a fusion chimera with a purification tag, such as an epitope tag, which is specifically bound by a purification tag-binding molecule utilized as the molecule-of-interest binding moiety of the molecular linker.

Such a crystallization system presents the advantage of enabling a single molecular linker to facilitate the crystallization of any polypeptide-of-interest, modified as described above.

All alternatives described in Example 7 above pertaining to functional segments of molecular linkers, and to methods of including metal atoms in crystallizable complexes are applicable to the presently disclosed method.

Production of a chimeric polypeptide comprising the molecule-of-interest and the tag is effected by cloning nucleic acid sequences encoding the molecule-of-interest into a bacterial expression vector which comprises a nucleic acid sequence encoding the tag, and which is configured to express the molecule-of-interest and the tag in-frame as a fusion protein.

Suitable bacterial strains are transformed with the expression vector, and recombinant chimera produced by transformants is recovered using standard recombinant protein technology, and is crystallized using standard crystallization conditions for X-ray crystallography.

Thus, this method provides a means of facilitating the crystallization and crystallographic analysis of a broad range of polypeptides of interest conjugated to a heterologous molecule via a single type of molecular linker.

### Example 9

### Generation of Crystals of G Protein Coupled Receptors Suitable for Determination of Three Dimensional Atomic Structure Thereof

A very large number of human diseases are associated with G protein coupled receptor disfunction, as illustrated by the fact that G protein-coupled receptors constitute the most prominent family of drug targets, as described above. Nevertheless, pharmacological treatment of diseases associated with GPCRs remains suboptimal, however. Thus, there is a very great need for novel GPCR specific drugs. One way to generate such drugs would be to elucidate the 3D atomic structure of GPCRs at high resolution so as to enable the rational design of pharmacological agents capable of having a desired regulatory effect on the activity of such receptors. However, prior art methods cannot be used to efficiently generate crystals of membrane proteins such as GPCRs, which crystals being suitable for determining the 3D atomic structure of such receptors at high resolution. In order to fulfill this important need, the present inventors have designed molecular linkers capable of being used to generate highly ordered, X-ray crystallography grade crystals of G protein coupled receptors suitable for X-ray crystallographic analysis of the 3D atomic structure of such receptors as follows.

Background:

Streptavidin: Streptavidin is a 159 amino acid residue protein produced by Streptomyces avidinii that binds up to four molecules of biotin with ultra-high affinity (K_{d}˜10⁻¹⁵ M; Green N M., 1990. Methods in Enzymology 184:51-67), to form an ultra-stable homotetramer that does not dissociate even in the presence of 6 M urea (Kurzban G P., 1991. J Biol Chem. 266, 14470-14477). The crystallographic structure of core streptavidin illustrates that each streptavidin monomer folds into an eight-stranded antiparallel β-barrel, with the biotin binding site built by residues of the barrel itself and a loop of an adjacent subunit to form a very stable dimer (Freitag S. et al., 1997. Protein Science 6:1157-1166). Extensive intersubunit contacts between the dimers give rise to the final tetrameric structure having tight quaternary assembly and fixed geometry (Green N M., 1990. Methods in Enzymology 184:51-67).

Another advantage of using streptavidin as the core of a molecular linker, is that extensive literature exists for the expression and purification of streptavidin itself (Wu S C. et al., 2002. Protein Expression and Purification 24:348-356; Gallizia A. et al., 1998. Protein Expression and Purification 14:192-196), and of streptavidin fusion proteins (Sano T. & Cantor C R. 2000. Methods Enzymol. 326:305-11). Smaller and more stable streptavidins than the native form have been produced recombinantly (Sano T. et al., 1993. Journal of Biological Chemistry 270:28204-28209) and the gene sequence has been optimized for expression in E. coli (Thompson L D. & Weber P C., 1993. Gene 136:243-6). The tetramer of these smaller cores displays enhanced stability under denaturing conditions, and their biotin binding sites appear to be more accessible. A small core size is also preferable, as it helps to keep the size of the final polypeptidic molecular linker to a minimum, making the scaffold easier and cheaper to produce and purify. Smaller molecular linkers may be advantageous since, as a rule of thumb, a smaller and tightly packed multimerization scaffolds will introduce less disorder in the final GPCR-linker complex, thus ensuring higher quality crystals.

Arrestins: The arrestin family consists of visual arrestin (v-arrestin, S-arrestin), cone-arrestin, β-arrestin (β-arrestin-1 and arrestin-2), and β-arrestin-2 (arrestin-3). V- and cone-arrestins are exclusively expressed in rod and cone photoreceptors, respectively, and are highly specialized to bind specifically to rhodopsin, or cone cell pigments. The two closely related β-arrestins are ubiquitously expressed and are responsible for the termination of the primary signaling event for most, if not all, class I (rhodopsin-like) GPCRs.

At the sequence level, visual arrestin is 60% identical to the β-arrestins, which show 78% sequence identity between themselves. The three dimensional structure of v-arrestin (Hirsch J A. et al., 1999. Cell 97:257-69; Granzin, J. et al., 1998. Nature 391:918-21) and of β-arrestin (Han M. et al., 2001. Structure (Camb) 9:869-80) have been solved and reported in the literature.

Arrestins bind with subnanomolar affinities (Gurevich V V. et al., 1995. Journal of Biological Chemistry 270:720-731) exclusively to agonist-activated GPCRs that have been phosphorylated by G protein-coupled receptor kinases (GRKs) on serine and threonine residues located in the third intracellular loop or carboxyl terminal tail (Gurevich V V. & Benovic J L., 1992. Journal of Biological Chemistry 267:21919-21923; Lohse M. et al., 1992. J Biol Chem. 267:8558-8564; Lohse M J. et al., 1990. Science 248:1547-50). The association of a single arrestin with a GRK-phosphorylated receptor uncouples the receptor from its cognate G protein, resulting in the termination of GPCR signaling, a process termed desensitization (Gurevich V V. & Benovic J L., 1992. Journal of Biological Chemistry 267:21919-21923; Lohse M. et al., 1992. J Biol Chem. 267:8558-8564; Lohse M J. et al., 1990. Science 248:1547-50; Pippig S. et al., 1993. Journal of Biological Chemistry 268:3201-3208; Attramadal H. et al., 1992. J Biol Chem. 267:17882-17890). In the case of β-arrestins, these molecules then target desensitized receptors to clathrin-coated pits for endocytosis by functioning as adaptor proteins that link the receptor to components of the endocytic machinery such as AP-2 and clathrin (Goodman, O B Jr. et al., 1996. Nature 383:447-50; Laporte S A. et al., 1999. Proc Natl Acad Sci USA. 96:3712-3717; Laporte S A. et al., 2000. J Biol Chem. 275:23120-23126; Ferguson S S G. et al., 1996. Science 271:363-366). The internalized receptors are dephosphorylated in endosomes and recycled back to the cell surface fully resensitized (Zhang L. et al., 1997. J Biol Chem. 272:14762-8; Oakley R H. et al., 1999. J Biol Chem. 274:32248-57; Krueger K M. et al., 1997. J Biol Chem 272:5-8).

The overall structures of β-arrestins and v-arrestin share many similar features: all are elongated molecules with a central polar core built by a network of charge-charge interactions (amino acid residues 1-8, 30, 175-176, 296, 303 and 382; where the numbering follows the sequence of v-arrestin) flanked by the N (amino acid residues 8-180) domain, C domain (amino acid residues 188-362) and a C tail (amino acid residues 372-404) that tightly interacts with the two domains and with the N terminus. Residues 98-108 in the N-domain form a cationic amphipathic α-helix that might serve as a reversible membrane anchor. Structural variations between arrestins are mostly found in surface loops. Analysis of β-arrestin and v-arrestin structures has shown that such arrestins are characterized by a very similar overall structure (Han M. et al., 2001. Structure (Camb) 9:869-80). The loop regions that vary between β-arrestin and v-arrestin also vary between different crystal forms of the same protein, reflecting the intrinsic flexibility of those regions rather than inherent structural differences between the two arrestins, as can be seen from the distribution of B factors. The crystal structures of v-arrestin and of β-arrestin analyzed represent their respective inactive basal states, where the polar core is intact.

It has been shown that the predominant region of receptor binding in v-arrestin is contained within amino acid residues 90-140. A portion of this region (amino acid residues 95-140) expressed as a fusion protein with glutathione S-transferase has been shown to be capable of binding to rhodopsin regardless of the activation or phosphorylation state of the receptor (Smith W C. et al., 1999. Biochemistry 38:2752-61). Mutations disrupting the polar core such as the v-arrestin mutant R175E, promote phosphorylation-independent binding of arrestin to the receptor (Gurevich V V. & Benovic J L. Molecular Pharmacology 51:161-169; GrayKeller M P. et al., 1997. Biochemistry 36:7058-7063). Segment-swapping experiments between visual and non-visual arrestins have demonstrated that substituting amino acid residues 50-90 of v-arrestin with the equivalent element of β-arrestin (amino acid residues 46-86) can switch the binding specificity of v-arrestin to high affinity binding of activation-phosphorylated m2 muscarinic cholinergic receptor (P-m2 mAchR*) while losing the affinity for activation-phosphorylated rhodopsin (P—Rh*; Han M. et al., 2001. Structure (Camb) 9:869-80). Remarkably, the single amino acid mutation V90S was shown to eliminate this difference, permitting v-arrestin to bind P-m2 mAchR* with similar affinity as β-arrestin without significant concurrent loss of its affinity to P—Rh*. In addition, elimination of the hydrophobic side chains of residues 11-13 was observed to disrupt the interaction between the N-domain and the amphipathic α-helix, and enhances phosphorylation-independent binding of arrestin (Vishnivetskiy S A. et al., 2000. J Biol Chem. 275:41049-41057).

These truncation and deletion studies point to the N-terminal domain as the primary domain of interaction—the truncated N-domain of arrestin binds to P-m2 mAchR with a K_{d}=2 nM (Gurevich V V. et al., 1995. Journal of Biological Chemistry 270:720-731). Additional data also point to the C-domain as playing a significant role in receptor binding since a truncated form of arrestin in which just a short C-terminal region is removed displays a K_{d}=1 nM (Gurevich V V. et al., 1995. Journal of Biological Chemistry 270:720-731).

The evidence accumulated so far suggest two possible mechanisms promoting receptor-arrestin interaction that are independent of the specific GPCR subtype. One mechanism is linked to the polar core, where critical salt bridges keep arrestin in its basal state (Hirsch J A. et al., 1999. Cell 97:257-69). An activation-phosphorylated GPCR would interact with arrestin, thereby disrupting the polar core and triggering the conformational changes required for high-affinity receptor binding. A second general mechanism can be derived from structural and mutagenesis data, whereby receptor binding is triggered and/or enhanced by the membrane translocation of arrestin's amphipathic α-helix I (Han M. et al., 2001. Structure (Camb) 9:869-80).

Materials and Methods:

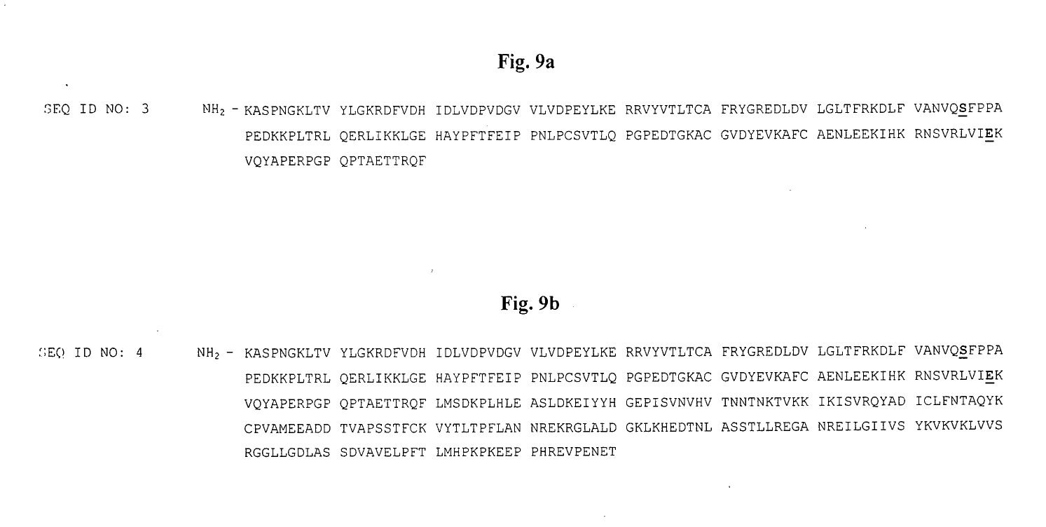

The above-described data relating to streptavidin indicates that core streptavidin can be used to generate molecular linkers having a highly stable and rigid predetermined quaternary structure and geometry suitable for optimally facilitating crystallization of crystallization complexes. The above-described data relating to arrestins indicates that a polypeptide composed of amino acid residues 11-190 of human beta-arrestin-1a with mutation R169E (SEQ ID NO: 3; FIG. 9a), or a polypeptide composed of amino acid residues 11-370 of human beta-arrestin-1a with mutation R169E (SEQ ID NO: 4; FIG. 9b) can serve as ligands capable of binding different classes of GPCRs with high affinity and specificity regardless of the phosphorylation/activation state thereof. Mutation R169E in human beta-arrestin-1a is homologous to the above-described R175E mutation in v-arrestin, as shown by published amino acid sequence comparisons (Han M. et al., 2001. Structure (Camb) 9:869-80; Hirsch J A. et al., 1999. Cell 97:257-69). Mutation R169E thus enables binding of GPCRs independently of the activation-phosphorylation state thereof. There is a serine residue located at position 86 in wild-type human beta-arrestin-1a which corresponds to mutation V90S in v-arrestin as shown by the aforementioned published amino acid sequence comparisons. As described hereinabove, the presence of a serine residue at this position confers the capacity to bind multiple types of GPCRs. Thus, the polypeptides corresponding to SEQ ID NOs: 3 and 4 have the capacity to bind multiple types of GPCRs as well as the capacity to bind GPCRs independently of the activation-phosphorylation state thereof. Thus, molecular linkers were designed incorporating a streptavidin based core and arrestin based GPCR binding portions.

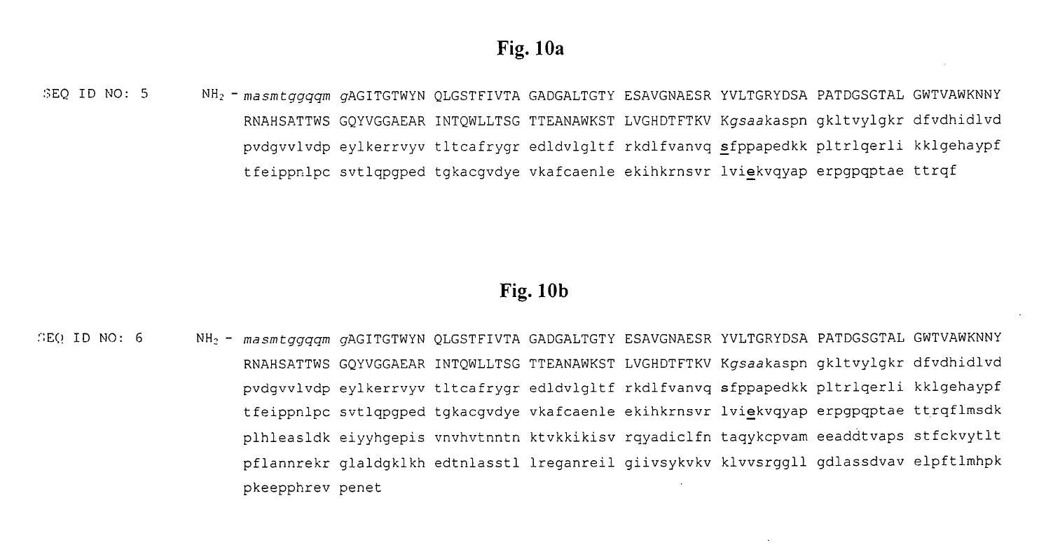

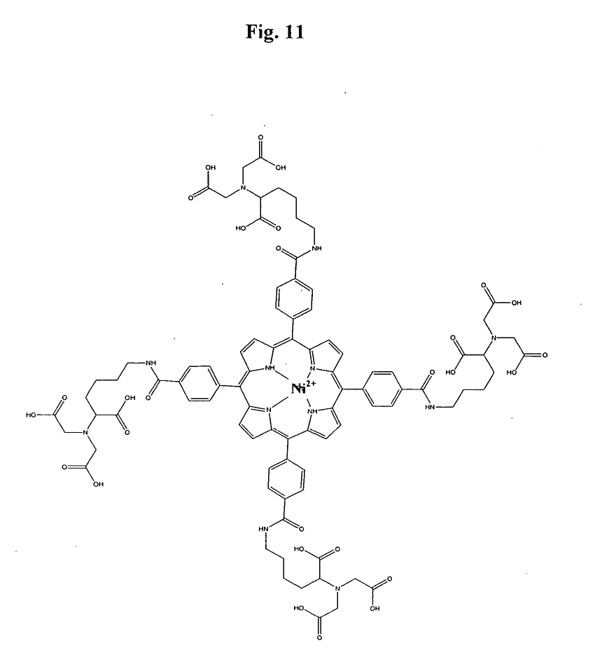

Streptavidin-arrestin chimera based molecular linkers: Two polypeptidic molecular linkers for generation of X-ray crystallography grade crystals of molecular linker-GPCR complexes were designed. The first linker (SEQ ID NO: 5; FIG. 10a) is composed of a chimeric protein consisting of the N- to C-terminal segments; T7 tag, core streptavidin, the peptide linker GSAA (SEQ ID NO: 1), and the above-described human beta-arrestin-1a derived polypeptide set forth in SEQ ID NO: 3. The second linker (SEQ ID NO: 6; FIG. 10b) is composed of a chimeric protein consisting of the N- to C-terminal segments; T7 tag, core streptavidin, the peptide linker GSAA (SEQ ID NO: 1), and the above-described human beta-arrestin-1a segment set forth in SEQ ID NO: 4.

These molecular linkers can be conjugated to a metal atom via biotinylated porphyrin synthesized, as described above. Molecular linkers having streptavidin cores can adopt a highly stable and rigid predetermined quaternary structure and geometry suitable for optimally facilitating crystallization of crystallization complexes, and bind with high specificity and affinity the largest possible set of different GPCRs.

Streptavidin-metallothionein chimera/arrestin-Strep-tag chimera based molecular linkers: Polypeptidic molecular linkers for generation of X-ray crystallography grade crystals of molecular linker-GPCR complexes were designed using a system of two polypeptide chimeras. One chimera consists of the N- to C-terminal segments; T7 tag, core streptavidin, and metallothionein. The other chimera consists of, the N- to C-terminal segments; the above-described human beta-arrestin-1a derived polypeptide set forth in SEQ ID NO: 3 or SEQ ID NO: 4 and a Strep-tag. In this system, the arrestin comprising chimera is attached to the core of the molecular linker by specific binding of the Strep-tag, to which the arrestin derived polypeptide is fused, to the core streptavidin contained in the molecular linker. The metallothionein segment can be used to incorporate several heavy metal atoms such as Cd²⁺ in the crystallization complex for providing initial phases for analysis of X-ray crystal diffraction data.

Metallothionein-streptavidin fusion proteins are produced essentially as previously described in the literature, with minor modifications for including the T7 tag and for adjusting the length of the streptavidin core (Sano T. et al., 1999. Proc Natl Acad Sci USA. 89:1534-8).

The T7 tag was used in order to increase production of recombinant proteins and to facilitate their purification.

The availability of the 3D structures of all proteins employed in the construction of the above-described polypeptidic molecular linkers has enabled modeling of the structure of such molecular linkers with a significant degree of confidence.

Chimeric proteins are cloned in standard expression vectors for expression of recombinant proteins in E. coli using standard recombinant DNA procedures on the basis of genomic DNA sequences, cDNA sequences or protein sequences of arrestins and streptavidins available in public and private databases (e.g., GenBank, EMBL, PIR, NCBI Pubmed, etc). Sequences coding for the fusion protein are codon-optimized for expression in E. coli (Thompson L D. & Weber P C., 1993. Gene 136:243-6). Streptavidin fusion proteins are optimally designed and produced with the streptavidin core at the N-terminus and are produced as inclusion bodies to maximize free biotin binding sites and refolding as previously described (Sano T. & Cantor C R. 2000. Methods Enzymol. 326:305-11). Introduction of the T7 tag at the N-terminus of the chimeric proteins increases expression thereof and permits easier purification thereof (Gallizia A. et al., 1998. Protein Expression and Purification 14:192-196. Recombinant chimeras are purified from bacterial inclusion bodies using standard techniques and T7 tag specific affinity chromatography. The purified molecular linkers are then individually mixed with different types of GPCRs at stoichiometric ratios, and under physiological conditions suitable for enabling complex formation therebetween. Formed complexes are subsequently subjected to crystallization inducing conditions.

For a one-step purification/molecular linker complexation procedure, fusion proteins containing core streptavidin, or molecular complexes containing such fusion proteins, are bound to affinity chromatography columns with matrices conjugated to streptavidin specific ligands, and are directly eluted from such columns using biotinylated molecular linker, such as biotinylated porphyrin (described above).

The monodispersity and second virial coefficient of solutions containing molecular linkers, GPCRs, and complexes comprising molecular linkers and/or GPCRs are monitored via light scattering techniques so as to select optimal preparations thereof for crystallization (Curtis R A. et al., 2001. Journal of Physical Chemistry B 105:2445-2452; Ruppert S. et al., 2001. Biotechnology Progress 17:182-187; Hitscherich C. et al., 2000. Protein Science 9:1559-1566).

Results:

With each of the above-described types of molecular linkers, different types of GPCRs are efficiently crystallized conjugated to heavy metal atoms suitable for generating initial phases for X-ray crystallographic analysis of 3D atomic structure. Such crystals are highly ordered, X-ray crystallography grade, crystals.

Conclusion: The above-described GPCR crystallization method can be used to generate highly purified, highly ordered, X-ray crystallography grade crystals of numerous classes of GPCRs, regardless of the activation/phosphorylation state thereof, suitable for determining the 3D atomic structure of such GPCRs. The present method is superior to all prior art methods, since prior art methods cannot be used to efficiently generate highly ordered crystals of different types of GPCRs.

### Example 10

### Efficient Purification of Different Classes of Correctly Folded G Protein-Coupled Receptors Via Arrestin Based Affinity Chromatography

As described in the previous Example, there is a vital need for novel GPCR targeting drugs. In order to provide the data required for producing such drugs, pharmacological, biochemical, and structural studies must be performed on GPCRs. Such studies require significant quantities of highly purified, correctly folded GPCRs. There is therefore a need for methods of producing large quantities of various types of correctly folded GPCRs. Various prior art approaches have been attempted for purifying GPCRs. One approach, has attempted isolating and purifying GPCRs from primary tissues. Another approach has attempted to isolate and purify GPCRs via expression of such molecules as recombinant proteins in heterologous systems. However, all prior art approaches are unsatisfactory for producing satisfactory yields of correctly folded GPCRs due to the low natural abundance of GPCRs in primary tissues, and due to the lack of a suitable method of purifying GPCRs, membrane proteins whose correct folding is highly dependent on the membranal environment, in the correctly folded state. Furthermore, prior art approaches cannot be used to efficiently purify multiple GPCR types. For example, purification tag based purification systems cannot discriminate between folded and unfolded states of tagged proteins, and furthermore are restricted by the requirement that the tag be accessible on the surface of the protein, and not buried within the protein. Affinity purification techniques based on monoclonal antibodies or specific receptor ligands require cumbersome testing and preparation, are expensive, and are typically dedicated to a single type of target molecule. Thus, all prior art approaches have failed to provide an adequate solution for efficient production of purified, correctly folded, GPCRs of various types. In order to fulfill this important need, the present inventors have devised a novel and improved method of isolating GPCRs as follows.

Materials and Methods:

The capacity of the above-described arrestin-derived polypeptides (SEQ ID NOs: 3 and 4) to bind numerous classes of GPCRs regardless of the activation-phosphorylation state thereof indicates that such polypeptides constitute ideal capture ligands for affinity chromatography of a wide range of GPCRs. Such forms of arrestin are used for affinity chromatography purification of GPCRs as follows.

Each of the above-described GPCR-binding human beta-arrestin-1a derived polypeptides (SEQ ID NOs: 3 and 4) is synthesized via standard recombinant protein production techniques, and is individually coupled to a suitable affinity purification support matrix such as an agarose, polyacrylamide, silica, cellulose or dextran matrix (Wilchek M. & Chaiken I., 2000. Methods Mol Biol 147:1-6; Jack, G W., 1994. Mol Biotechnol. 1:59-86; Narayanan S R., 1994. Journal of Chromatography A 658:237-258; Nisnevitch M. & Firer M A., 2001. J Biochem Biophys Methods 49:467-80; Janson J C. & Kristiansen T. in Packings and Stationary Phases in Chromatography Techniques (ed. Unger, K. K.) 747 (Marcel Dekker, New York, 1990)).

The GPCR-binding polypeptides are coupled to the support matrix covalently and in an orientation specific manner via a standard coupling reaction (see, for example: Wilchek M. & Chaiken I., 2000. Methods Mol Biol 147:1-6; Jack G W., 1994. Mol Biotechnol. 1:59-86; Narayanan S R., 1994. Journal of Chromatography A 658:237-258; Nisnevitch M. & Firer M A., 2001. J Biochem Biophys Methods 49:467-80; Clonis Y D. in HPLC of Macromolecules A Practical Approach 157 (IRL Press, Oxford, 1989)).

Alternatively, GPCR-binding polypeptides are produced fused to a Strep-tag (Schmidt T G M. et al., 1996. Journal of Molecular Biology 255:753-766; Skerra A. & Schmidt T G M., 1999. Biomolecular Engineering 16:79-86), as previously described (Nilsson J. et al., 1997. Protein Expr Purif. 11:1-16), and is coupled to a support matrix conjugated to streptavidin.

As a further alternative, the arrestin segment is produced fused to an N-terminal core streptavidin moiety and is a coupled to a support matrix conjugated with Strep-tag peptide or iminobiotin (Sano T. et al., 1998. Journal of Chromatography B 715:85-91).

An affinity chromatography column is prepared using the arrestin-conjugated matrix, a sample containing a soluble GPCR is applied to the column, the column is subjected to a cycle of washes for removal of contaminants, and fractions are eluted using a suitable buffer. Free GPCR is then eluted using a buffer containing a peptide that specifically competes with GPCR for binding with arrestin (Gurevich V V. et al., 1995. Journal of Biological Chemistry 270:720-731; Smith, W. C. et al., 1999. Biochemistry 38:2752; Raman D. et al., 1999. Biochemistry 38:5117-23; Bennett T A. et al., 2001. J Biol Chem. 276:22453-60; Sternemarr R. et al., 1993. Journal of Biological Chemistry 268:15640-15648); tagged arrestin-GPCR complex is eluted using a standard buffer specific for uncoupling the tag from its matrix-conjugated ligand (Nilsson J. et al., 1997. Protein Expr Purif. 11:1-16); or streptavidin-arrestin fusion protein is eluted with biotin, or a biotinylated molecule, such as biotinylated porphyrin, as described in the preceding Example, thereby enabling simultaneous purification and molecular linker complexation thereof. Elution of GPCR as a complex with the arrestin ligand is advantageous for obtaining correctly folded GPCR in high yield due to arrestin functioning as a stabilizing adjuvant to the receptor preparation (Hulme E C. & Curtis C A., 1998. Biochemical Society Transactions 26:S361) Separation of GPCR from tagged arrestin is then effected using the aforementioned peptide that specifically competes with the GPCR for binding with arrestin.

Purification of GPCR in eluted fractions is monitored via standard light scattering techniques.

The above described procedure is repeated using different classes of unmodified or suitably modified GPCRs using the same type of, or the same suitably recycled, purification column.

Results:

Significant quantities of highly purified, correctly folded GPCRs of numerous classes are produced.

Conclusion: The above-described method of the present invention can be used conveniently and rapidly produce large quantities of highly purified, correctly folded GPCRs of different classes. Such purified GPCRs can be used to obtain valuable information required for generating novel GPCR-targeting drugs. As such, the method of the present invention is significantly superior to prior art methods which cannot be used to efficiently purify various types of correctly folded GPCRs in significant quantities.

### Example 11

### Universal Molecular Linkers for Crystallization of Histidine-Tagged Membrane Proteins

Solution of the 3D structure of membrane proteins, is crucial for the rational design of drugs targeting such proteins. To date, X-ray diffraction analysis of highly ordered crystals comprising such proteins remains the only way to solve the 3D atomic structure of such proteins. However, no prior art crystallization methods can be used to efficiently generate such crystals. In order to fulfill the critical need for such methods, the present inventors have devised universal molecular linkers for crystallizing essentially any histidine tagged membrane protein.

Materials and Methods:

Crystallization via porphyrin-NTA-Ni²⁺ molecular linker: A porphyrin based molecular linker comprising N-(5-amino-1-carboxypentyl)imino-diacetic acid (NTA) groups is synthesized and is chelated to Ni²⁺ using standard chemical techniques. A schematic diagram of porphyrin-NTA-Ni²⁺ molecular linker is shown in FIG. 11. A sample containing a recombinant histidine tagged membrane protein displaying an accessible histidine tag is generated using standard techniques (e.g., refer to Sheibani N., 1999. Prep Biochem Biotechnol. 29:77). The sample containing the histidine-tagged membrane protein is reacted with porphyrin-NTA-Ni²⁺ in the appropriate stoichiometry and under suitable reaction conditions for formation of complexes of porphyrin-NTA-Ni²⁺ and the histidine-tagged protein. Complexation occurs via association of the chelated nickel ion with the histidine tag of the membrane protein. The complex is purified, dissolved in a suitable buffer, and is crystallized using standard crystallization conditions.

The above described process is repeated using different histidine-tagged membrane proteins.

Crystallization via anti histidine tag single-chain Fv-core streptavidin fusion protein molecular linker: In order to crystallize a membrane protein-of-interest, a polypeptidic molecular linker composed of a fusion protein comprising, from N- to C-terminal; anti histidine tag single chain Fv derived from monoclonal antibody 3D5 (Kaufmann, M. et al., 2002. J Mol Biol. 318. 135-47) and core streptavidin is generated. The recombinant single chain Fv-core streptavidin chimera is produced as previously described, with minor modifications (see, for example: Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188). The membrane protein-of-interest is produced as a recombinant histidine tagged protein displaying an accessible histidine tag using standard techniques (e.g., refer to Sheibani N. 1999. Prep Biochem Biotechnol. 29:77). A sample containing the histidine-tagged membrane protein-of-interest is reacted with the single chain Fv-core streptavidin molecular linker in an appropriate stoichiometry under suitable reaction conditions for formation of complexes of the molecular linker and the histidine-tagged protein (refer, for example to: Kaufmann, M. et al., 2002. J Mol Biol. 318. 135-47). The complex is purified, dissolved in a suitable buffer, and is crystallized using standard crystallization conditions.

Results:

Highly ordered, X-ray crystallography grade crystals, each containing a different membrane protein, are efficiently generated using both porphyrin-NTA and anti histidine tag single-chain Fv-core streptavidin based molecular linkers.

Conclusions: The above-described molecular linkers can be used to efficiently generate different highly ordered, X-ray crystallography grade crystals, each comprising a different membrane protein. Such crystals can be used to determine the 3D atomic structure of such membrane proteins. As such the method of the present invention is superior to all prior art methods of generating membrane proteins since these cannot be used to efficiently generate highly ordered crystals of membrane proteins.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of generating a crystal containing a molecule-of-interest, the method comprising:
(a) contacting molecules of the molecule-of-interest with at least one type of heterologous molecular linker being capable of interlinking at least two molecules of the molecule-of-interest to thereby form a crystallizable molecular complex of defined geometry; and
(b) subjecting said crystallizable molecular complex to crystallization-inducing conditions, thereby generating the crystal containing the molecule-of-interest.

2. The method of claim 1, wherein said at least one type of heterologous molecular linker is selected such that said crystallizable molecular complex formed is capable of generating a crystal selected from the group consisting of a 2D crystal, a helical crystal and a 3D crystal.

3. The method of claim 1, wherein the molecule-of-interest is a polypeptide.

4. The method of claim 3, wherein said polypeptide is a membrane protein.

5. The method of claim 4, wherein said membrane protein is a G protein coupled receptor.

6. The method of claim 5, wherein said G protein coupled receptor is rhodopsin or is a class A G protein coupled receptor.

7. The method of claim 6, wherein said class A G protein coupled receptor is m2 muscarinic cholinergic receptor.

8. The method of claim 1, wherein said at least one type of heterologous molecular linker includes a region for specifically binding the molecule-of-interest.

9. The method of claim 8, wherein the molecule-of-interest is a G protein coupled receptor and whereas said region for specifically binding the molecule-of-interest comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

10. The method of claim 9, wherein said at least a portion of an arrestin molecule is homologous to amino acid residues 11 to 190, or 11 to 370 of human beta-arrestin-1a.

11. The method of claim 9, wherein said at least a portion of an arrestin molecule comprises a G protein coupled receptor-binding domain of said arrestin molecule.

12. The method of claim 9, wherein said mutation at an amino acid residue position corresponding to position 90 in bovine visual arrestin is a mutation to a serine or threonine residue.

13. The method of claim 9, wherein said mutation at an amino acid residue position corresponding to position 175 in bovine visual arrestin is a mutation to a glutamic acid or an asparagine residue.

14. The method of claim 9, wherein said G protein coupled receptor is rhodopsin or is a class A G protein coupled receptor.

15. The method of claim 14, wherein said class A G protein coupled receptor is m2 muscarinic cholinergic receptor.

16. The method of claim 8, wherein the molecule-of-interest includes a histidine tag and whereas said region for specifically binding the molecule-of-interest comprises a nickel ion or an antibody specific for said histidine tag.

17. The method of claim 8, wherein the molecule-of-interest includes core streptavidin and whereas said region for specifically binding the molecule-of-interest comprises a biotin moiety or a Strep-tag.

18. The method of claim 8, wherein the molecule-of-interest includes a biotin moiety or a Strep-tag and whereas said region for specifically binding the molecule-of-interest comprises core streptavidin.

19. The method of claim 1, wherein the molecule-of-interest is a G protein coupled receptor and whereas said at least one type of molecular linker comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

20. The method of claim 19, wherein said at least a portion of an arrestin molecule is homologous to amino acid residues 11 to 190, or 11 to 370 of human beta-arrestin-1a.

21. The method of claim 9, wherein said at least a portion of an arrestin molecule comprises a G protein coupled receptor-binding domain of said arrestin molecule.

22. The method of claim 19, wherein said mutation at an amino acid residue position corresponding to position 90 in bovine visual arrestin is a mutation to a serine or threonine residue.

23. The method of claim 19, wherein said mutation at an amino acid residue position corresponding to position 175 in bovine visual arrestin is a mutation to a glutamic acid or an asparagine residue.

24. The method of claim 19, wherein said G protein coupled receptor is rhodopsin or is a class A G protein coupled receptor.

25. The method of claim 24, wherein said class A G protein coupled receptor is m2 muscarinic cholinergic receptor.

26. The method of claim 1 wherein said at least one type of heterologous molecular linker includes at least two non-covalently bound subunits.

27. The method of claim 26, wherein said at least two non-covalently bound subunits comprise a first subunit comprising a homomultimerizing portion and a metal-binding portion, and a second subunit comprising a portion specifically binding the molecule-of-interest, and a portion specifically binding said first subunit.

28. The method of claim 26, wherein said at least two non-covalently bound subunits comprise a first subunit comprising a homomultimerizing portion and a portion specifically binding the molecule-of-interest, and a second subunit comprising a metal-binding portion, and a portion specifically binding said first subunit.

29. The method of claim 1, wherein said at least one type of heterologous molecular linker includes a molecule selected from the group consisting of a polycyclic molecule, a polydentate ligand, a macrobicyclic cryptand, a polypeptide and a metal.

30. The method of claim 1, wherein said at least one type of heterologous molecular linker comprises core streptavidin.

31. The method of claim 1, wherein said at least one type of heterologous molecular linker is selected so as to define the spatial positioning and orientation of said at least two molecules within said crystallizable molecular complex, thereby facilitating crystallization of the molecule-of-interest.

32. The method of claim 1, wherein said at least one type of heterologous molecular linker includes a hydrophilic region, said hydrophilic region being for facilitating crystallization of the molecule-of-interest.

33. The method of claim 1, wherein said at least one type of heterologous molecular linker includes a conformationally rigid region, said conformationally rigid region being for facilitating crystallization of the molecule-of-interest.

34. The method of claim 1, wherein said at least one type of heterologous molecular linker includes a metal-binding moiety capable of specifically binding a metal atom, said metal atom being capable of facilitating crystallographic analysis of the crystal.

35. The method of claim 34, wherein said metal-binding moiety is a metal binding protein.

36. The method of claim 35, wherein said metal binding protein is metallothionein.

37. The method of claim 1, wherein said at least one type of heterologous molecular linker includes a region being capable of functioning as a purification tag, said purification tag being capable of facilitating purification of said crystallizable molecular complex and/or of facilitating said interlinking at least two molecules of the molecule-of-interest.

38. The method of claim 37, wherein said region being capable of functioning as a purification tag is selected from the group consisting of a T7 tag, a histidine tag, a Strep-tag, core streptavidin, and biotin.

39. The method of claim 1, wherein the molecule-of-interest includes a region being capable of functioning as a purification tag, said purification tag being capable of facilitating purification of said crystallizable molecular complex, and/or of facilitating said interlinking at least two molecules of the molecule-of-interest.

40. The method of claim 39, wherein said region being capable of functioning as a purification tag is selected from the group consisting of a T7 tag, a histidine tag, a Strep-tag, core streptavidin, and biotin.

41. The method of claim 1, wherein the molecule-of-interest includes a metal-binding moiety capable of specifically binding a metal atom, said metal atom being capable of facilitating crystallographic analysis of the crystal.

42. The method of claim 41, wherein said metal-binding moiety is a metal binding protein.

43. The method of claim 42, wherein said metal binding protein is metallothionein.

44. A method of generating a crystal containing a polypeptide of interest, the method comprising:
(a) providing a molecule including the polypeptide of interest and a heterologous multimerization domain being capable of directing the homomultimerization of the polypeptide of interest;
(b) subjecting said molecule to homomultimerization-inducing conditions, thereby forming a crystallizable molecular complex; and
(c) subjecting said crystallizable molecular complex to crystallization-inducing conditions, thereby generating the crystal containing the polypeptide of interest.

45. The method of claim 44, wherein (a) and (b) are effected concomitantly.

46. The method of claim 44, wherein said heterologous multimerization domain is selected such that said crystallizable molecular complex formed is capable of generating a crystal selected from the group consisting of a 2D crystal, a helical crystal and a 3D crystal.

47. The method of claim 44, wherein said heterologous multimerization domain includes a hydrophilic region, said hydrophilic region being for facilitating crystallization of the polypeptide of interest.

48. The method of claim 44, wherein said heterologous multimerization domain includes a conformationally rigid region, said conformationally rigid region being for facilitating crystallization of the polypeptide of interest.

49. The method of claim 44, wherein said heterologous multimerization domain is selected so as to define the spatial positioning and orientation of polypeptides of the polypeptide of interest within said crystallizable molecular complex, thereby facilitating crystallization of the polypeptide of interest.

50. The method of claim 44, wherein said heterologous multimerization domain comprises core streptavidin.

51. The method of claim 44, wherein the polypeptide of interest is a G protein coupled receptor and whereas said heterologous multimerization domain comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

52. The method of claim 51, wherein said at least a portion of an arrestin molecule is homologous to amino acid residues 11 to 190, or 11 to 370 of human beta-arrestin-1a.

53. The method of claim 52, wherein said at least a portion of an arrestin molecule comprises a G protein coupled receptor-binding domain of said arrestin molecule.

54. The method of claim 51, wherein said mutation at an amino acid residue position corresponding to position 90 in bovine visual arrestin is a mutation to a serine or threonine residue.

55. The method of claim 51, wherein said mutation at an amino acid residue position corresponding to position 175 in bovine visual arrestin is a mutation to a glutamic acid or an asparagine residue.

56. The method of claim 5 1, wherein said G protein coupled receptor is rhodopsin or is a class A G protein coupled receptor.

57. The method of claim 56, wherein said class A G protein coupled receptor is m2 muscarinic cholinergic receptor.

58. The method of claim 44, wherein the polypeptide of interest includes a histidine tag and whereas said heterologous multimerization domain comprises a nickel ion or an antibody specific for said histidine tag.

59. The method of claim 44, wherein the polypeptide of interest includes core streptavidin and whereas said heterologous multimerization domain comprises a biotin moiety or a Strep-tag.

60. The method of claim 44, wherein the polypeptide of interest includes a biotin moiety or a Strep-tag and whereas said heterologous multimerization domain comprises core streptavidin.

61. The method of claim 44, wherein the polypeptide of interest and said heterologous multimerization domain are interlinked via a molecular linker.

62. The method of claim 61, wherein at least one of said heterologous multimerization domain and said molecular linker include a hydrophilic region, said hydrophilic region being for facilitating crystallization of the polypeptide of interest.

63. The method of claim 61, wherein at least one of said heterologous multimerization domain and said molecular linker include a conformationally rigid region, said conformationally rigid region being for facilitating crystallization of the polypeptide of interest.

64. The method of claim 61, wherein at least one of said heterologous multimerization domain and said molecular linker is selected so as to define the spatial positioning and orientation of polypeptides of the polypeptide of interest within said crystallizable molecular complex, thereby facilitating crystallization of the polypeptide of interest.

65. The method of claim 61, wherein said at least one molecular linker includes a region being capable of functioning as a purification tag, said purification tag being capable of facilitating purification of said crystallizable molecular complex, and/or of facilitating said homomultimerization of the polypeptide of interest.

66. The method of claim 65, wherein said region being capable of functioning as a purification tag is selected from the group consisting of a T7 tag, a histidine tag, a Strep-tag, core streptavidin, and biotin.

67. The method of claim 44, wherein the polypeptide of interest includes a region being capable of functioning as a purification tag, said purification tag being capable of facilitating purification of said crystallizable molecular complex, and/or of facilitating said homomultimerization of the polypeptide of interest.

68. The method of claim 67, wherein said region being capable of functioning as a purification tag is selected from the group consisting of a T7 tag, a histidine tag, a Strep-tag, core streptavidin, and biotin.

69. The method of claim 44, wherein said molecule includes a metal-binding moiety capable of specifically binding a metal atom, said metal atom being capable of facilitating crystallographic analysis of the crystal.

70. The method of claim 69, wherein said metal-binding moiety is a metal binding protein.

71. The method of claim 70, wherein said metal binding protein is metallothionein.

72. The method of claim 44, wherein the polypeptide of interest is a membrane protein.

73. The method of claim 72, wherein said membrane protein is a G protein coupled receptor.

74. The method of claim 73, wherein said G protein coupled receptor is rhodopsin or is a class A G protein coupled receptor.

75. The method of claim 74, wherein said class A G protein coupled receptor is m2 muscarinic cholinergic receptor.

76. The method of claim 44, wherein the polypeptide of interest includes a metal-binding moiety capable of specifically binding a metal atom, said metal atom being capable of facilitating crystallographic analysis of the crystal.

77. The method of claim 70, wherein said metal binding moiety is metallothionein.

78. A composition-of-matter comprising at least two molecules of a molecule-of-interest interlinked via a heterologous molecular linker, wherein said heterologous molecular linker is selected so as to define the relative spatial positioning and orientation of said at least two molecules within the composition-of-matter, thereby facilitating formation of a crystal therefrom under crystallization-inducing conditions.

79. The composition-of-matter of claim 78, wherein the molecule-of-interest is a polypeptide.

80. The composition-of-matter of claim 79, wherein said polypeptide is a membrane protein.

81. The composition-of-matter of claim 80, wherein said membrane protein is a G protein coupled receptor.

82. The composition-of-matter of claim 81, wherein said G protein coupled receptor is rhodopsin or is a class A G protein coupled receptor.

83. The composition-of-matter of claim 82, wherein said class A G protein coupled receptor is m2 muscarinic cholinergic receptor.

84. The composition-of-matter of claim 78, wherein said heterologous molecular linker includes at least one region capable of specifically binding said molecule-of-interest.

85. The composition-of-matter of claim 84, wherein said molecule-of-interest is a G protein coupled receptor and whereas said at least one region capable of specifically binding said molecule-of-interest is a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

86. The composition-of-matter of claim 85, wherein said at least a portion of an arrestin molecule is homologous to amino acid residues 11 to 190, or 11 to 370 of human beta-arrestin-1a.

87. The composition-of-matter of claim 86, wherein said at least a portion of an arrestin molecule comprises a G protein coupled receptor-binding domain of said arrestin molecule.

88. The composition-of-matter of claim 85, wherein said mutation at an amino acid residue position corresponding to position 90 in bovine visual arrestin is a mutation to a serine or threonine residue.

89. The composition-of-matter of claim 85, wherein said mutation at an amino acid residue position corresponding to position 175 in bovine visual arrestin is a mutation to a glutamic acid or an asparagine residue.

90. The composition-of-matter of claim 85, wherein said G protein coupled receptor is rhodopsin or is a class A G protein coupled receptor.

91. The composition-of-matter of claim 90, wherein said class A G protein coupled receptor is m2 muscarinic cholinergic receptor.

92. The composition-of-matter of claim 78, wherein said heterologous molecular linker includes a molecule selected from the group consisting of a polycyclic molecule, a polydentate ligand, a macrobicyclic cryptand, a polypeptide and a metal.

93. The composition-of-matter of claim 78, wherein said molecule-of-interest is a G protein coupled receptor and whereas said heterologous molecular linker comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, SEQ ID NO: 4, SEQ ID NO: 5, and SEQ ID NO: 6.

94. The composition-of-matter of claim 93, wherein said at least a portion of an arrestin molecule is homologous to amino acid residues 11 to 190, or 11 to 370 of human beta-arrestin-1a.

95. The composition-of-matter of claim 94, wherein said at least a portion of an arrestin molecule comprises a G protein coupled receptor-binding domain of said arrestin molecule.

96. The composition-of-matter of claim 93, wherein said mutation at an amino acid residue position corresponding to position 90 in bovine visual arrestin is a mutation to a serine or threonine residue.

97. The composition-of-matter of claim 93, wherein said mutation at an amino acid residue position corresponding to position 175 in bovine visual arrestin is a mutation to a glutamic acid or an asparagine residue.

98. The composition-of-matter of claim 93, wherein said G protein coupled receptor is rhodopsin or is a class A G protein coupled receptor.

99. The composition-of-matter of claim 98, wherein said class A G protein coupled receptor is m2 muscarinic cholinergic receptor.

100. The composition-of-matter of claim 78, wherein said heterologous molecular linker comprises core streptavidin.

101. The composition-of-matter of claim 78, wherein said heterologous molecular linker includes at least two non-covalently bound subunits.

102. The composition-of-matter of claim 78, wherein said heterologous molecular linker includes a hydrophilic region, said hydrophilic region being for facilitating crystallization of said molecule-of-interest.

103. The composition-of-matter of claim 78, wherein said heterologous molecular linker includes a conformationally rigid region, said conformationally rigid region being for facilitating crystallization of said molecule-of-interest.

104. The composition-of-matter of claim 78, wherein said heterologous molecular linker is selected such that the composition-of-matter is capable of generating a crystal selected from the group consisting of a 2D crystal, a helical crystal and a 3D crystal.

105. The composition-of-matter of claim 78, wherein said heterologous molecular linker includes a metal-binding moiety capable of specifically binding a metal atom, said metal atom being capable of facilitating crystallographic analysis of the crystal.

106. The composition-of-matter of claim 105, wherein said metal-binding moiety is a metal-binding protein.

107. The composition-of-matter of claim 106, wherein said metal binding protein is metallothionein.

108. The composition-of-matter of claim 78, wherein said heterologous molecular linker includes a region being capable of functioning as a purification tag, said purification tag being capable of facilitating purification of the crystallizable composition-of-matter, and/or of facilitating said interlinking of said at least two molecules of a molecule-of-interest.

109. The composition-of-matter of claim 78, wherein said region being capable of functioning as a purification tag is selected from the group consisting of a T7 tag, a histidine tag, a Strep-tag, core streptavidin, and biotin.

110. The composition-of-matter of claim 78, wherein said molecule-of-interest includes a region being capable of functioning as a purification tag, said purification tag being capable of facilitating purification of the composition-of-matter, and/or of facilitating said interlinking of said at least two molecules of a molecule-of-interest.

111. The composition-of-matter of claim 110, wherein said region being capable of functioning as a purification tag is selected from the group consisting of a T7 tag, a histidine tag, a Strep-tag, core streptavidin, and biotin.

112. The composition-of-matter of claim 78, wherein said molecule-of-interest includes a metal-binding moiety capable of specifically binding a metal atom, said metal atom being capable of facilitating crystallographic analysis of the crystal.

113. The composition-of-matter of claim 112, wherein said metal-binding moiety is a metal binding protein.

114. The composition-of-matter of claim 113, wherein said metal-binding protein is metallothionein.

115. A nucleic acid construct comprising a polynucleotide segment encoding a chimeric polypeptide including:
(a) a first polypeptide region being capable of specifically binding a molecule-of-interest; and
(b) a second polypeptide region being capable of specifically binding a metal atom.

116. The nucleic acid construct of claim 115, wherein said molecule-of-interest is a G protein coupled receptor and whereas said chimeric polypeptide comprises SEQ ID NO: 5 or SEQ ID NO: 6.

117. The nucleic acid construct of claim 116, wherein said G protein coupled receptor is rhodopsin or is a class A G protein coupled receptor.

118. The nucleic acid construct of claim 117, wherein said class A G protein coupled receptor is m2 muscarinic cholinergic receptor.

119. The nucleic acid construct of claim 115, wherein said molecule-of-interest is a G protein coupled receptor and whereas said first polypeptide region comprises a molecule selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

120. The nucleic acid construct of claim 119, wherein said at least a portion of an arrestin molecule is homologous to amino acid residues 11 to 190, or 11 to 370 of human beta-arrestin-1a.

121. The nucleic acid construct of claim 120, wherein said at least a portion of an arrestin molecule comprises a G protein coupled receptor-binding domain of said arrestin molecule.

122. The nucleic acid construct of claim 119, wherein said mutation at an amino acid residue position corresponding to position 90 in bovine visual arrestin is a mutation to a serine or threonine residue.

123. The nucleic acid construct of claim 119, wherein said mutation at an amino acid residue position corresponding to position 175 in bovine visual arrestin is a mutation to a glutamic acid or an asparagine residue.

124. The nucleic acid construct of claim 119, wherein said G protein coupled receptor is rhodopsin or is a class A G protein coupled receptor.

125. The nucleic acid construct of claim 124, wherein said class A G protein coupled receptor is m2 muscarinic cholinergic receptor.

126. The nucleic acid construct of claim 115, wherein the molecule-of-interest is a polypeptide.

127. The nucleic acid construct of claim 126, wherein said polypeptide is a membrane protein.

128. The nucleic acid construct of claim 127, wherein said membrane protein is a G protein coupled receptor.

129. The nucleic acid construct of claim 128, wherein said G protein coupled receptor is rhodopsin or is a class A G protein coupled receptor.

130. The nucleic acid construct of claim 129, wherein said class A G protein coupled receptor is m2 muscarinic cholinergic receptor.

131. The nucleic acid construct of claim 115, wherein said second polypeptide region is metallothionein.

132. The nucleic acid construct of claim 115, wherein said chimeric polypeptide is selected such that when combined with molecules of said molecule-of-interest under suitable conditions, said chimeric polypeptide and said molecules form a crystallizable molecular complex which is capable of forming a crystal containing said molecule-of-interest when subjected to crystallization-inducing conditions.

133. The nucleic acid construct of claim 115, wherein said chimeric polypeptide is selected such that when combined with molecules of said molecule-of-interest and said metal atom under suitable conditions, said chimeric polypeptide and said molecules form a crystallizable molecular complex which is capable of forming a crystal containing said molecule-of-interest when subjected to crystallization-inducing conditions.

134. The nucleic acid construct of claim 132, wherein said metal atom facilitates crystallographic analysis of said crystal.

135. The nucleic acid construct of claim 132, wherein said chimeric polypeptide includes a hydrophilic region, said hydrophilic region being for facilitating crystallization of said molecule-of-interest.

136. The nucleic acid construct of claim 132, wherein said chimeric polypeptide includes a conformationally rigid region, said conformationally rigid region being for facilitating crystallization of said molecule-of-interest.

137. The nucleic acid construct of claim 132, wherein said chimeric polypeptide is selected so as to define the spatial positioning and orientation of said molecule-of-interest within said crystallizable molecular complex, thereby facilitating crystallization of said molecule-of-interest.

138. The nucleic acid construct of claim 132, wherein said chimeric polypeptide is selected such that said crystallizable molecular complex formed is capable of generating a crystal selected from the group consisting of a 2D crystal, a helical crystal and a 3D crystal.

139. The nucleic acid construct of claim 132, wherein said chimeric polypeptide further includes a polypeptide region being capable of functioning as a purification tag, said purification tag being capable of facilitating purification of said crystallizable molecular complex, and/or of facilitating said binding of a molecule-of-interest.

140. The nucleic acid construct of claim 139, wherein said region being capable of functioning as a purification tag is selected from the group consisting of a T7 tag, a histidine tag, a Strep-tag, core streptavidin. and biotin.

141. A nucleic acid construct comprising a polynucleotide segment encoding a chimeric polypeptide including:
(a) a first polypeptide region being capable of specifically binding a molecule-of-interest;
(b) a second polypeptide region being capable of homomultimerization into a complex of defined geometry; and
(c) a third polypeptide region being capable of specifically binding a metal atom.

142. The nucleic acid construct of claim 141, wherein said molecule-of-interest is a G protein coupled receptor and whereas said first polypeptide region is selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, SEQ ID NO: 3, and SEQ ID NO: 4.

143. The nucleic acid construct of claim 142, wherein said at least a portion of an arrestin molecule is homologous to amino acid residues 11 to 190, or 11 to 370 of human beta-arrestin-1a.

144. The nucleic acid construct of claim 143, wherein said at least a portion of an arrestin molecule comprises a G protein coupled receptor-binding domain of said arrestin molecule.

145. The nucleic acid construct of claim 142, wherein said mutation at an amino acid residue position corresponding to position 90 in bovine visual arrestin is a mutation to a serine or threonine residue.

146. The nucleic acid construct of claim 9, wherein said mutation at an amino acid residue position corresponding to position 175 in bovine visual arrestin is a mutation to a glutamic acid or an asparagine residue.

147. The nucleic acid construct of claim 142, wherein said G protein coupled receptor is rhodopsin or is a class A G protein coupled receptor.

148. The nucleic acid construct of claim 147, wherein said class A G protein coupled receptor is m2 muscarinic cholinergic receptor.

149. The nucleic acid construct of claim 141, wherein said second polypeptide region comprises core streptavidin.

150. The nucleic acid construct of claim 141, wherein said molecule-of-interest is a G protein coupled receptor and whereas said chimeric polypeptide comprises SEQ ID NO: 5 or SEQ ID NO: 6.

151. The nucleic acid construct of claim 150, wherein said G protein coupled receptor is rhodopsin or is a class A G protein coupled receptor.

152. The nucleic acid construct of claim 151, wherein said class A G protein coupled receptor is m2 muscarinic cholinergic receptor.

153. The nucleic acid construct of claim 141, wherein said third polypeptide region comprises metallothionein.

154. The nucleic acid construct of claim 141, wherein the molecule-of-interest is a polypeptide.

155. The nucleic acid construct of claim 154, wherein said polypeptide is a membrane protein.

156. The nucleic acid construct of claim 155, wherein said membrane protein is a G protein coupled receptor.

157. The nucleic acid construct of claim 156, wherein said G protein coupled receptor is rhodopsin or is a class A G protein coupled receptor.

158. The nucleic acid construct of claim 157, wherein said class A G protein coupled receptor is m2 muscarinic cholinergic receptor.

159. The nucleic acid construct of claim 141, wherein said chimeric polypeptide is selected such that when combined with molecules of said molecule-of-interest, said chimeric polypeptide and said molecules form a crystallizable molecular complex of defined geometry which is capable of forming a crystal containing said molecule-of-interest when subjected to crystallization-inducing conditions.

160. The nucleic acid construct of claim 159, wherein said chimeric polypeptide includes a hydrophilic region, said hydrophilic region being for facilitating crystallization of said molecule-of-interest.

161. The nucleic acid construct of claim 159, wherein said chimeric polypeptide includes a conformationally rigid region, said conformationally rigid region being for facilitating crystallization of said molecule-of-interest.

162. The nucleic acid construct of claim 159, wherein said chimeric polypeptide is selected so as to define the spatial positioning and orientation of molecules of said molecule-of-interest within said crystallizable molecular complex, thereby facilitating crystallization of said molecule-of-interest.

163. The nucleic acid construct of claim 159, wherein said chimeric polypeptide is selected such that said crystallizable molecular complex of defined geometry formed is capable of generating a crystal selected from the group consisting of a 2D crystal, a helical crystal and a 3D crystal.

164. The nucleic acid construct of claim 159, wherein said metal atom facilitates crystallographic analysis of said molecule-of-interest contained in said crystal.

165. The nucleic acid construct of claim 159, wherein said chimeric polypeptide further includes a polypeptide region being capable of functioning as a purification tag, said purification tag being capable of facilitating purification of said crystallizable molecular complex, and/or of facilitating said binding of a molecule-of-interest.

166. The nucleic acid construct of claim 165, wherein said region being capable of functioning as a purification tag is selected from the group consisting of a T7 tag, a histidine tag, a Strep-tag, and core streptavidin.

167. A method of purifying a G protein coupled receptor from a sample containing the G protein coupled receptor, the method comprising subjecting the sample to affinity chromatography using an affinity ligand selected from the group consisting of at least a portion of an arrestin molecule, at least a portion of an arrestin molecule having a mutation at an amino acid residue position corresponding to position 90 in bovine visual arrestin, at least a portion of an arrestin molecule having a mutation at an amino acid residue position corresponding to position 175 in bovine visual arrestin, a molecule defined by SEQ ID NO: 3, and a molecule defined by SEQ ID NO: 4, thereby purifying the G protein coupled receptor.

168. The method of claim 167, wherein said at least a portion of an arrestin molecule is homologous to amino acid residues 11 to 190, or 11 to 370 of human beta-arrestin-1a.

169. The method of claim 168, wherein said at least a portion of an arrestin molecule comprises a G protein coupled receptor-binding domain of said arrestin molecule.

170. The method of claim 167, wherein said mutation at an amino acid residue position corresponding to position 90 in bovine visual arrestin is a mutation to a serine or threonine residue.

171. The method of claim 167, wherein said mutation at an amino acid residue position corresponding to position 175 in bovine visual arrestin is a mutation to a glutamic acid or an asparagine residue.

172. The method of claim 167, wherein said G protein coupled receptor is rhodopsin or is a class A G protein coupled receptor.

173. The method of claim 172, wherein said class A G protein coupled receptor is m2 muscarinic cholinergic receptor.

174. The method of claim 167, wherein said affinity ligand includes a region being capable of functioning as a purification tag, said purification tag being capable of facilitating attachment of said affinity ligand to an affinity chromatography matrix.

175. The method of claim 174, wherein said region being capable of functioning as a purification tag is selected from the group consisting of a T7 tag, a histidine tag, a Strep-tag, core streptavidin, and biotin.


SEQUENCE LISTING (6 sequences)

SEQ ID NO: 1 - protein, 4 aa, Artificial sequence
  A peptide linker for crystallization of different classes of GPCRs
GSAA

SEQ ID NO: 2 - protein, 2 aa, Artificial sequence
  A peptide linker for crystallization of different classes of GPCRs
GS

SEQ ID NO: 3 - protein, 180 aa, Homo sapiens
KASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIEKVQYAPERPGPQPTAETTRQF

SEQ ID NO: 4 - protein, 360 aa, Homo sapiens
KASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIEKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPENET

SEQ ID NO: 5 - protein, 315 aa, Artificial sequence
  A polypeptidic molecular linker for generation of X-ray crystallography grade crystal of molecular linker-GPCR complexes
MASMTGGQQMGAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKGSAAKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIEKVQYAPERPGPQPTAETTRQF

SEQ ID NO: 6 - protein, 495 aa, Artificial sequence
  A polypeptidic molecular linker for generation of X-ray crystallography grade crystal of molecular linker-GPCR complexes
MASMTGGQQMGAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKGSAAKASPNGKLTVYLGKRDFVDHIDLVDPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIEKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPENET